(19) United States
(12) Patent Application Publication
(10) Pub. No.: US 2008/0026986 A1
(43) Pub. Date: Jan. 31, 2008

(54) REVERSAL OF THE SUPPRESSIVE FUNCTION OF SPECIFIC T CELLS VIA TOLL-LIKE RECEPTOR 8 SIGNALING

(76) Inventors: Rong-Fu Wang, Houston, TX (US); Guangyong Peng, Houston, TX (US); Yicheng Wang, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 11/757,235

(22) Filed: Jun. 1, 2007

Related U.S. Application Data
(60) Provisional application No. 60/811,037, filed on Jun. 5, 2006.

(51) Int. Cl. A61K 31/7042; A61K 38/02; A61P 35/00

(52) U.S. Cl. 514/2; 514/44

(57) ABSTRACT

CD8⁺ regulatory T (Treg) cells and γδ Treg cells profoundly suppress host immune responses and thus protect against autoimmune disease while restricting desired immune responses such as antitumor immunity. Synthetic phosphorothioate-protected, guanosine-containing oligonucleotides can directly reverse the suppressive activity of Treg cells without involving dendritic cells. This effect appears to be transduced by signaling through Toll-like receptor (TLR) 8 and engagement of the MyD88 and IRAK4 molecules in Treg cells, in specific embodiments. Stimulation of Treg cells with natural ligands for human TLR8 recapitulated the effect of the synthetic guanosine-containing oligonucleotides.

### CROSS-REFERENCE TO RELATED APPLICATIONS

This Application claims the benefit under 35 U.S.C. 119(e) of U.S. Provisional Patent Application 60/811,037 filed on Jun. 5, 2006, the contents of which are hereby incorporated by reference in their entirety.

### STATEMENT REGARDING FEDERALLY SPONSORED RESEARCH OR DEVELOPMENT

This invention was made in part with government support from the National Institutes of Health under Grant Nos. R01 CA94327, R01 CA101795, P01CA90327, and P50 CA093459. The United States Government may have certain rights in the invention.

### BACKGROUND OF THE INVENTION

Immunotherapy affords a promising approach to the treatment of various types of cancer (Old, 1996; Rosenberg, 2001; Houghton et al., 2001; Wang, 2002; Arlen et al., 2006; McNeel and Malkovsky, 2005). Although peptide- or dendritic cell (DC)-based vaccines can induce antigen-specific immune responses, objective clinical responses remains infrequent and transient (McNeel and Malkovsky, 2005; Rosenberg, 2004). One explanation is that tumor cells may create an immune suppressive environment in cancer patients. Thus, a better understanding of the interaction between tumor-infiltrating immune cells and cancer cells is critical to efforts to devise strategies that would enhance the therapeutic efficacy of immunological interventions.

Recent studies indicate that preexisting CD4⁺ regulatory T (Treg) cells at tumor sites may pose major obstacles to effective cancer immunotherapy, as these cells have a potent ability to suppress host immune responses (Wang et al., 2004; Wang et al., 2005; Baecher-Allan and Anderson, 2006). Indeed, increased proportions of CD4⁺ CD25⁺ Treg cells in the total CD4⁺ T cell populations have been documented in patients with different types of cancers, including lung, breast and ovarian tumors (Woo et al., 2001; Curiel et al., 2004; Wang et al., 2006). The recent findings further demonstrate the presence of antigen-specific CD4⁺ Treg cells at tumor sites, where they induce antigen-specific and local immune tolerance (Wang et al., 2004; Wang et al., 2005). The removal or elimination of Treg cell populations with anti-CD25 monoclonal antibody (mAb) treatment results in effective rejection of transplanted tumors in animal models (Onizuka et al., 1999; Jones et al., 2002), further indicating a functional role for these Treg cells in tumor progression and immune suppression.

Since Treg cell-mediated immune suppression exists at tumor sites, a new strategy for depletion of Treg cells or reversal of the suppressive function of Treg cells will be important in efforts to induce antigen-specific effector T cells. Thus, the inventors recently demonstrated that Toll-like receptor (TLR) 8 ligands can specifically reverse the suppressive function of both antigen-specific and naturally occurring Treg cells (Peng et al., 2005). Treatment of Treg cells with polyguanosine oligonucleotides (poly-G) enhanced antitumor immunity in an animal model, but whether TLR8 signaling pathway can also control the suppressive function of other regulatory T cells, such as CD8⁺ Treg and γδ⁺ Treg cells was heretofore unknown.

Thus, T cells play an essential role in immunosurveillance and destruction of cancer cells, but this knowledge has not yielded clinically effective immunotherapies (Dunn et al., 2004; Rosenberg et al., 2004). It is generally thought that the major impediment to effective immunotherapy is the presence of Treg cells at tumor sites, which significantly suppress immune responses and induce immune tolerance (Woo et al., 2001; Liyanage et al., 2002; Wang et al., 2004; Curiel et al., 2004; Wang et al., 2005). Although CD4⁺ Treg cells have been extensively studied, much less is known about other subsets of Treg cells including γδ TCR T cells, which may function as regulatory T cells and suppress immune responses (Shevach , 2002; Sakaguchi, 2004; Hayday and Tigelaar, 2003).

γδ TCR T cells represent a small subset (2-3%) of T cells in the total T cell population, consisting of γ and δ TCR chains with limited TCR usage. In clear contrast to recognition of antigens by αβ T cells, γδ T cells recognize antigens directly without antigen processing/presentation or major histocompatibility complex (MHC) molecules (Brenner et al., 1986; Shin et al., 2005). Of two major subsets of human γδ T cells, γ2Vδ2 or γ9Vδ2 (referred to as Vδ2) T cells predominate in the peripheral blood and respond to bacterial and viral infections by recognizing small nonpeptidic molecules (such as isopentenyl pyrophosphate and alkyl amines) (Modlin et al., 1989; Constant et al., 1994; Bukowski et al., 1999). Recent studies demonstrated that human γ9δ2 T cells recognize endogenous mevalonate metabolites, phosphoantigens and F1-ATPase expressed by tumor cells (Fisch et al., 1990; Gober et al., 2003; Kabelitz et al., 2005; Viey et al., 2005; Scotet et al., 2005). The other major subset, Vδ1T cells, represent 70-90% of the T cells in the epithelial tissues (also called intraepithelial lymphocytes, IELs) and recognize MICA and/or MICB that are induced on epithelial cells and tumor cells by stress or damage (Groh et al., 1998; Hayday, 2000). MICA and some distantly related ULBP proteins are ligands for NKG2D, an activating NK receptor expressed on γδ T cells, NK cells and some αβ T cells (Bauer et al., 1999). In contrast to human γδ⁺ T cells, murine dendritic epidermal γδT cells (DETCs) do not recognize bacterial phosphoantigens, but recognize mycobacterial heat shock proteins, inducible MHC class Ib molecules T10/T22 and stress-related Rae-1 and H60 molecules (Diefenbach et al., 2000; Cerwenka et al., 2000). These MHC class I related molecules are expressed in transformed or tumor cells, thus stimulating antitumor immunity (Groh et al., 1999; Diefenbach et al., 2001), thus raising the possibility that recognition of MICA/B molecules expressed on transformed or tumor cells might afford a new strategy by which one could exploit the innate immune system to develop more effective cancer immunotherapy (Hayday and Tigelaar, 2003; Boismenu and Havran, 1994; Jameson et al., 2002; Girardi et al., 2001). Recent studies further demonstrated the diverse function of γδ T cells, showing that human Vδ2 T cells function as professional antigen-presenting cells (APCs) to elicit αβ T cell responses (Brandes et al., 2005).

Despite the important roles of γδ T cells as a natural component of host innate immunity in the surveillance of stressed or damaged tissues, malignancy and infectious pathogens (Hayday and Tigelaar, 2003; Havran, 2000; Jameson et al., 2003), whether these γδ T cells have the potent ability to suppress immune responses remains largely unknown (Seo et al., 1999; Ke et al., 2003; Kapp et al., 2004). Murine skin γδ T cells suppressed pro-inflammatory immune responses and prevented dermatitis in adoptive transfer experiments (Shiohara et al., 1990; Shiohara et al., 1996). Moreover, TCRγ-deficient mice in non-obese diabetic (NOD) background developed spontaneous cutaneous inflammation (dermatitis) (Girardi et al., 2002). These studies imply that γδ⁺ T cells may negatively regulate immune responses, but direct evidence for their function and regulatory mechanisms is still lacking.

Therefore, there is a need in the art to provide methods and compositions to reverse the suppressive function of at least CD8⁺ and γδ T regulatory cells.

### SUMMARY OF THE INVENTION

The invention relates to a method for inhibiting or modulating the immunosuppressive capacity of particular T cells, such as CD8⁺ T reg cells or γδ T reg cells, for example. In specific embodiments, the particular CD8⁺Treg cells may be further defined as being CD8⁺CD25⁺, CD3⁺, FoxP3⁺, GITR⁺, while γδ T reg cells do not have specific markers. In particular aspects of the invention, the inhibition of immunosuppressive capacity of particular T cells allows for improving efficacy of a therapy for a particular medical condition, such as cancer, infectious disease, or autoimmune disease, for example. Such methods may be respectively considered to be methods of increasing an anti-cancer response (such as an anti-humor response, for example) or for increasing an anti-infectious disease response.

The immunosuppressive activity of the T cells in the individual may be inhibited by any suitable composition, but in specific embodiments of the invention the immunosuppressive activity is at least partially inhibited by delivering one or more TLR8 ligands to the individual. In certain aspects of the invention, the immunosuppressive activity is at least partially inhibited by short guanine-comprising oligonucleotides. In specific embodiments, the immunosuppressive activity of the T cells is inhibited by targeting at least part of the TLR8-IRKA4-MyD88 signal transduction pathway. The oligonucleotides may be further defined as comprising a guanosine and a partially stabilized or nuclease-resistant inter-residue backbone. The oligonucleotide may also be further defined as comprising a nuclease-resistant inter-residue linkage between a guanosine and an adjacent residue.

In certain embodiments of the invention, there is at least one method for identifying compounds that inhibit the immunosuppressive capacity of an exemplary CD8⁺ and/or γδ Treg cell. In specific embodiments, the method comprises a comparison of cellular growth and/or division rates of parallel samples of naïve respective CD8⁺ T cells or γδ T cells. In particular, naïve CD8⁺ or γδ T cells exposed to uninhibited Treg cells are compared to control respective naïve CD8⁺ T cells or γδ T cells and respective naïve CD8⁺ T cells or γδ T cells exposed to Treg cells treated with a candidate compound. The reversal of Treg suppression is measured by the relative growths of the variously treated respective naïve cells, which may be CD8⁺ or γδ T cells. In further embodiments, the invention comprises delivery of the one or more identified inhibitory compounds to decrease Treg cell mediated immunosuppression in the context of an organism in need thereof, such as one in need of augmenting an antigen-specific immune response, for example an individual in need of increasing an antigen-specific immune response to an infection and/or cancer. The resultant increase in immune activity facilitates the organism's immune response to combat the disease state.

In certain embodiments, the methods of the present invention prevent immunosuppression by T cells. For example, an individual susceptible to having cancer or at risk for developing cancer (such as being a smoker, having a family history, having a personal history, having benign growths, and so forth) or becoming infected with an infectious disease is subjected to one or more methods and/or compositions of the present invention to prevent or delay onset of respectively having cancer and/or contracting the infectious disease.

In specific embodiments of the invention, there is demonstration of CD8⁺ regulatory T cells and their functional reversal by TLR8 signaling in prostate cancer. In particular, it is shown that the majority (70%) of prostate tumor-infiltrating T lymphocytes (PTILs) analyzed contained elevated proportions of CD4⁺ CD25⁺ T cells in the total T-cell population. Besides CD4⁺ T cells, the CD8⁺ T cell subpopulation also had potent suppressive activity. T-cell cloning analysis confirmed the presence of CD4⁺ CD25⁺FoxP3⁺ and CD8⁺ CD25⁺ FoxP3⁺ Treg cell clones in bulk PTIL lines. These Treg cells suppress immune responses mainly through a cell contact-dependent mechanism, although some inhibited naïve T cell proliferation via unknown soluble factors (other than IL-10 and TGF-β). The suppressive function of Treg cells could be reversed by human Toll-like receptor 8 (TLR8) signaling, regardless of the subsets represented and the suppressive mechanisms operative in Treg cells. These results indicate that Treg cells play a role in the induction of immune tolerance at prostate tumor sites and that the reversal of their suppressive function by TLR8 ligands improves the efficacy of immunotherapy for prostate cancer, in particular aspects of the invention.

In other specific embodiments, the present invention concerns tumor-infiltrating γδ regulatory T cells and their functional regulation in cancer, for example breast cancer. In particular, in recent efforts by the inventors to establish tumor-specific T cells from breast cancers, it was unexpectedly found γδ 1 T cells represented a dominant population in the total Tumor-infiltrating lymphocytes (TILs), in a sharp contrast to 2-3% γδ1 T cells in normal tissue-infiltrating lymphocytes. This prompted the inventors to further characterize tumor-specific γδ1 T cells for their function and regulatory mechanisms. In the present invention, there is described breast cancer-specific γδ1 T cells isolated from a breast cancer patient and prevalence of γδ1 T cells in both breast and prostate tumor samples surgically removed from cancer patients. Tumor-specific γδ1 T cells suppressed naïve T cell proliferation and inhibited IL-2 release from CD4⁺ and CD8⁺ effector cells. They also blocked the maturation and function of dendritic cells (DCs), suggesting that these γδ1 T cells function as γδ Treg cells. Although these γδ Treg cells were capable of killing autologous tumor cells, but failed to kill T cells, DCs nor other cell lines. Their ability to kill tumor cells required antigen-specific activation and was mediated by TRAIL-dependent pathway. Finally, the suppressive effects of γδ1 T cells on naïve/effector T cells could be reversed by TLR8 signaling. By contrast, treatment of γδ1 Treg cells with TLR8 ligands did not affect their killing ability of tumor cells, indicating that their suppressive function was not coupled to tumor cell killing.

Thus, in one embodiment of the invention there is a method for suppressing the activity of a CD8+ or γδ T regulatory cell comprising providing to the cell an effective amount of a composition capable of suppressing the activity of the T regulatory cell, wherein the composition is not a Type D CpG oligonucleotide. In a specific embodiment, the composition is further defined as a toll-like receptor 8 (TLR8) ligand, and the composition may be further defined as an oligonucleotide, such as an oligonucleotide further defined as a non CpG containing oligonucleotide, which oligonucleotide may comprise between about 4 and about 15 nucleotide residues or between about 5 and about 10 nucleotide residues. In specific embodiments, the oligonucleotide comprises at least one guanine and at least one nuclease-resistant inter-residue backbone linkage and may further comprise a nuclease-sensitive inter-residue backbone linkage. In particular aspects, the oligonucleotide comprises a nuclease resistant inter-residue backbone linkage connecting the guanine to an adjacent nucleobase. In additional specific embodiments, the cell is within a subject, such as a human. In specific aspects, the method further comprises providing the human with a therapeutic agent, such as an anti-cancer agent, an anti-bacterial agent, an anti-immune disease agent, or an anti-viral agent.

In another embodiment of the invention, there is a method for suppressing the activity of at least one CD8+ or γδ T-regulatory cell, comprising providing to the cell an effective amount of at least one recombinant DNA capable of activating the TLR8-MyD88-IRAK4 signal transduction pathway in the cell. In specific embodiments, the recombinant DNA is not a type-D CpG oligonucleotide, and the recombinant DNA may be further defined as a non CpG containing recombinant DNA such as, for example, one that comprises between about 4 and about 15 nucleotide residues or between about 5 and about 10 nucleotide residues.

The recombinant DNA may comprise at least one guanine residue and at least one nuclease-resistant inter-residue backbone linkage, and it may further comprise a nuclease-sensitive inter-residue backbone linkage. In a further specific embodiment, at least one guanine residue has at least one nuclease resistant inter-residue backbone linkage connecting the guanine residue with an adjacent nucleotide. The cell may be within an organism, such as a mammal, and including a human. The method may further comprise providing the human with a therapeutic agent, such as an anti-cancer agent, an anti-bacterial agent, an anti-immune disease agent, or an anti-viral agent.

In an additional embodiment, there is a method of treating an organism with an immune-related disease, comprising administering to said organism an effective amount of at least one recombinant DNA capable of modulating or suppressing the activity of at least one CD8+ or γδ T-regulatory cell, thereby increasing an immune response, wherein the recombinant DNA is not a type D CpG oligonucleotide. In a specific embodiment, said immune-related disease comprises cancer, infectious disease, or autoimmune disease. The recombinant DNA may be further defined as a non CpG containing recombinant DNA, such as one that comprises between about 4 and about 15 nucleotide residues, or between about 5 and about 10 nucleotide residues. In a specific embodiment, the recombinant DNA comprises at least one guanine residue and at least one nuclease-resistant inter-residue backbone linkage. In a further specific embodiment, at least one guanine residue has at least one nuclease resistant inter-residue backbone linkage connecting the guanine residue with an adjacent nucleotide. The organism may be a mammal, such as a human, and the method may further comprising providing the human with a therapeutic agent, such as, for example, an anti-cancer agent, an anti-bacterial agent, an anti-immune disease agent, or an anti-viral agent.

In a further embodiment of the invention, there is a method for screening for compounds that inhibit the suppressive function of Treg cells, comprising the steps of: a. subjecting a Treg cell to a candidate compound; b. stimulating the proliferation of a naïve T cell; c. exposing the naïve T cell to the Treg cell; and d. determining the degree of growth or proliferation of the naïve T cell. In a specific embodiment, the candidate compound is selected from a library of candidate compounds and may be, for example, an oligonucleotide, a polypeptide, a polynucleotide, a small molecule, or a mixture thereof. In specific embodiments, the degree of proliferation is measured relative to the degree of proliferation of a control, the control consisting essentially of a Treg cell exposed to an oligonucleotide incapable of suppressing Treg cell activity. In a specific embodiment, the candidate compound is suspected of being a TLR8 ligand.

In another embodiment of the invention, there is a method for screening for compounds that inhibit the suppressive function of Treg cells, comprising the steps of: providing Treg cells in the presence of naïve T cells; subjecting said Treg cells to a candidate compound; and assessing proliferation of the naïve T cells, wherein when there is proliferation of the naïve T cells as compared to that in the presence of the Treg cells but absence of the candidate compound, said candidate compound is said compound that inhibits suppressive function of Treg cells. In a specific embodiment, the candidate compound is suspected of being a TLR8 ligand and may be an oligonucleotide, a polypeptide, a polynucleotide, a small molecule, or a mixture thereof.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention. All articles, papers and other references cited herein are incorporated by reference. This incorporation by reference includes the articles, papers and other references listed within or otherwise cited by these incorporated articles, papers and other references.

### BRIEF DESCRIPTION OF THE DRAWINGS

For a more complete understanding of the present invention, reference is now made to the following descriptions taken in conjunction with the accompanying drawings.

FIGS. 1A-1C show FACS and functional analysis of bulk TILs derived from prostate cancers. (A) Low percentages of CD4⁺ CD25⁺ T cells in PTIL120, PTIL123 and PTIL128. Prostate tumor-derived TILs were stained with FITC-conjugated anti-CD4 mAb and PE-conjugated anti-CD25 mAb; healthy donor-derived T cells served as a control. PTIL120, PTIL123 and PTIL128 contained low percentages of CD4⁺ CD25⁺ T cells, and did not suppress the proliferation of naive CD4⁺ T cells. (B) High percentages of CD4⁺ CD25⁺ T cells in PTIL157, PTIL194, PTIL237 and PTIL313. These PTILs suppressed the proliferative response of naive CD4⁺ T cells, while control CD4⁺ T cells did not. (C) Analysis of CD4⁺CD25⁺ T cells in melanoma-derived TILs. Most melanoma-derived TILs contained low percentages of CD4⁺CD25⁺ T cells, and did not suppress the proliferative response of naive CD4⁺ T cells.

FIG. 2 shows functional analysis of CD4⁺ and CD8⁺ T-cell populations in bulk TILs. CD4⁺ or CD8⁺ T cells were purified from bulk PTIL lines with a bead-coated anti-CD4 or anti-CD8 antibody (left panels). The purity of CD4⁺ or CD8⁺ T cells was more than 95%. The suppressive function of each CD4⁺ or CD8⁺ T cell population was tested for their ability to suppressive naïve T cell proliferation (right panels).

FIGS. 3A-3C show identification and characterization of Treg cells in bulk PTIL194 (A) Generation of Treg cell clones with suppressive activity. T-cell clones were screened for their suppressive activity in a proliferation assay. Fifty-one clones showed strong suppressive activity, while 43 clones did not or weakly suppressed the proliferation of naive CD4⁺ T cells. (B) Determination of FoxP3 expression levels in PTIL194 and PTIL237 by real-time PCR. HPRT served as an internal control. (C) Phenotypic analysis of T-cell clones by FACS. CD4⁺ T cell clones and CD8⁺ T-cell clones without suppressive activity served as controls for Treg cell clones with suppressive activity.

FIGS. 4A-4B show cell contact-dependent inhibition by PTIL194 and PTIL237. (A) Cell-cell contact is required for Treg suppression. Equal numbers of CD4⁺ responding T cells were cultured in the outer wells, while PTIL194 or PTIL237 were cultured in the inner wells of a transwell plate. Cocultured cells served as the positive control for PTIL194 and PTIL237. (B) There was no detectable suppressive activity of the responding CD4⁺ T cells in the outer wells, once separated from CD4⁺ or CD8⁺ Treg cell clones generated from PTIL194 in the inner well.

FIGS. 5A-5C show reversal of the suppressive function of PTIL194 and PTIL237 by TLR ligands. (A) Restoration of Treg cell suppressed proliferation of naïve CD4⁺ T cells by TLR8 ligands (poly-G2, CpG-A and ssRNA40). Ligands for other TLRs, including loxoribine, LPS, poly(I:C), pam3CSK4, fragellin and imiquimod, showed no effect on naïve T-cell proliferation (B) Reversal of suppressive function of CD4⁺ Treg and CD8⁺ Treg cell clones by poly-G2. (C) Expression of TLR8 mRNA in CD4⁺ Treg and CD8⁺ Treg cell clones generated from PTIL194 by RT-PCR.

FIGS. 6A-6D show generation and characterization of tumor-specific γδ₁ T cells. (A) Recognition of autologous breast tumor cells by breast cancer-derived BTIL31 cells. Tumor reactivity of T cells was determined by IFN-γ release from T cells. Other cell lines of allogeneic breast cancer cell lines, prostate cancer cell lines, melanoma cell lines and EBV-B cell line or 293-derived cell lines served as controls for specificity of T cell recognition. (B) Antigen specificity of BTIL31-derived T cell clones. Similar experiments were performed as in (A), but with T cell clones derived from the bulk BTIL31 T cells. (C) FACS analysis of surface markers of BTIL31 cells. BTIL31 cells were stained with phycoerythrin (PE)- or FITC-labeled mAb to CD3, CD4, CD8, CD56, CD161, TCR-αβ and TCR-γδ molecules. Isotype control antibodies served as negative controls. (D) BTIL31 bulk and clones cells were predominantly γδ₁T cells. BTIL31 cells and clones were stained with phycoerythrin (PE)- or FITC-labeled mAb to TCR-γδ, TCR-Vδ1, TCR-Vδ2 and TCR-Vδ9.

FIGS. 7A-7C show prevalence and suppressive function of breast and prostate tumor-derived γδ T cells. (A) High proportions of γδ T cells in breast tumor-derived BTILs. BTILs were stained with PE-labeled anti-CD8, FITC-labeled anti-CD4 and anti-TCR-γδ mAbs. (B) High percentages of γδ cells in prostate tumor-derived PTILs, but low percentages in melanoma-derived MTILs. (C) Functional analysis of BTILs. Both γδ⁺ T cells and γδ⁻ (i.e. CD4⁺ and CD8⁺) T cells were purified by FACS sorting and used to test their ability to inhibit naïve T cell proliferation. Both γδ⁺ T cells and γδ⁻ T cells in 4 of 6 BTILs have strongly suppressive activity on the proliferation of naïve CD4⁺ T cells.

FIGS. 8A-8D show regulatory property of BTIL31 and its clones. (A) Suppression of naïve T cell proliferation by BTIL31 and its clone cells. The proliferation of naïve CD4⁺ (responding) T cells (1×10⁵/well) were inhibited by different number of BTIL31 cells and clones C1-C4 in the presence of anti-CD3 antibody. By contrast, naïve CD4⁺ T cells and γδ T cells freshly purified from PBMCs of healthy dornors enhanced the proliferation of responding CD4⁺ T cells. (B) BTIL31 and its clones inhibited the ability of 1363-C1 helper cells to secrete IL-2 after stimulation with 1363mel target cells. Anti-CD3 antibody activated BTIL31 cells and clones were cocultured with CD4⁺ TIL1363-C1 helper cells for 24 hours. After washing, 1363mel tumor cells were added to according mixture of TILs. IL-2 secretion in the culture supernatants was determined by ELISA after 18 hours incubation. BTIL31 and its clones strongly inhibited the ability of 1363-C1 helper cells to secret IL-2. By contrast, Naïve CD4⁺ T cells (control) did not affect the ability of 1363-C1 helper cells to secret IL-2. Results are one representative data of three independent experiments. (C) Immunosuppression of naïve T cell proliferation by BTIL31 cells does not require a cell-cell contact mechanism. The freshly purified naive CD4⁺ T cells were cultured in the outer wells, while equal numbers of BTIL31 cells and its clones or naive CD4⁺ T cells served as a control were added into the inner wells in the same medium as outer wells. The proliferation of the cells in the outer and inner wells were detected by [³H]-thymidine incorporation assay. BTIL31 and its clones inhibited the proliferation of naïve CD4⁺ T cells in the outer wells, but control CD4⁺ T cells did not have detectable suppressive activity. Results are one representative data set from three independent experiments. (D) Culture supernatants of BTIL31 and its clones were capable of suppressing naïve T cell proliferation. The culture supernatants from BTIL31 and its clones or naïve CD4⁺0 T cells served as a control were added to the proliferation assay cultures of naïve CD4⁺ T cells in total 200 μl reaction volumes. Only 10 μl supernatants from BTIL31 or its clones completely inhibited the proliferation of naïve CD4⁺ T cells, while control supernatants from naïve CD4⁺ T cells even augmented the proliferation. Data from one of three independent experiments with similar results are shown.

FIGS. 9A-9C provide cytokine profiles and phenotypic analyses of BTIL31 γδT cells. (A) Cytokine profiles of BTIL31 and its clones. BTIL31 and its clones were cocultured with BC31 autologous tumor cells for 18-24 h, and cytokines of IFN-γ, GM-CSF, IL-2, IL-4, IL-10 and TGF-β in the culture supernatants were determined by ELISA. The data are means ⁺SEM; error bars indicate the standard deviation (n=4). (B) Phenotypic marker analysis. BTIL31 cells did not express CD25 and GITR markers. (C) A low expression level of Foxp3 in BTIL31 and its clones. Foxp3 expression in BTIL31 and its clones was determined by real-time quantitative PCR analysis using primers and an internal fluorescent proble for Foxp3 or HPRT (Hypoxanthine-guanine phosphoribosyl-transferase). The relative quantity of Foxp3 in each sample was normalized to the relative quantity of HPRT. Control CD4⁺ Treg 102-C3 and Treg 164-C2 express high level Foxp3. CD4⁺ effector TIL1363-5B10 served as a negative control. Results in (B) and (C) are one representative set experiments of three independent experiments.

FIGS. 10A-10C provide inhibitory effects of BTIL31 and its clones on DC maturation and function. (A) Inhibition of DC maturation by BTIL31 cells. The treated and untreated DCs were harvested and stained with PE or FITC-labeled anti-CD83, CD80, CD86 and HLA-DR antibodies, and analyzed by FACScan. BTIL31-treated DCs strongly down-regulated the expression of CD83, CD80, CD86 and HLA-DR, while naïve CD4⁺ T cells-treated DCs had no effect on the expression of these molecules. (B) Impairment of DC's ability to secrete IL-6 and IL-12 by BTIL31 cells in response to LPS stimulation. The procedure and cell culture condition are indicated. After 48 h culture, the treated and untreated DCs were harvested and transferred to 96-well plates and stimulated with LPS (5 μg/ml) for 24 hrs. IL-12 and IL-6 release in the culture supernatants was detected by ELISA Kit. (C) Impairment of DC's ability to stimulate naïve T cell proliferation in the presence of soluble OKT3 antibody after treated with BTIL31 cells in a transwell. The immature and mature DCs were treated as indication, and transferred to 96-well plate to test capacity of stimulation on the proliferation of naïve CD4⁺ T cells. 1×10⁵ allogeneic naïve CD4⁺ T cells were cocultured with different numbers of treated or untreated DCs, and the proliferation of allogeneic naïve CD4⁺ T cells were determined by [³H]-thymidine incorporation assay described as above. Results in (A) to (C) are one representative data of three independent experiments.

FIGS. 11A-11D demonstrate killing of autologous tumor cells, but not T cells, DCs or melanoma, by BTIL31 cells through a TRAIL pathway. (A) BTIL31 T cells killed BC31 breast tumor cells, but not 586LCL, 1363mel, DCs, CD4⁺ effector or naïve T cells. The same number of carboxyfluorescein diacetate succinimidyl ester (CFSE) labeled different of target cells (CFSE, 4.5 μM) and control 1558mel cells (CFSE, 0.5 μM) were cocultured with BTIL31 cells at a 1:1 ratio in 24-well plates. 12-15 h later, the cells were harvested and analyzed by FACS gating on the CFSE-labeled cells. BTIL31 cells only killed the autologous tumor cells but not other target cells. (B) The killing ability of BTIL31 T cells could be completely blocked by an anti-TCR-γδ antibody, and partially inhibited by an anti-NK-G2D antibody, but not by control antibody or antibodies against MHC class I, class II, MICA/B and CD1d molecules. BTIL31-C1 cells were cocultured with BC31 autologous tumor cells in the presence or absence of all kinds of blocking antibodies, and IFN-γ secretion in the culture supernatant was determined after 18-24 h incubation. (C) Transfection of BC31 cells with a MICA cDNA enhanced T cell recognition. 293T and BC29 cells with or without transfection of MICA served as controls. Transfected and untransfected cells were cocultured with BTIL31 cells, and IFN-γ secretion in the culture supernatant was determined after 18-24 h incubation. (D) Inhibition of tumor cell killing by an anti-TRAIL antibody. The equal number of BC31 tumor cells or BTIL31-C1 cells were mixed in the absence or presence of anti-FasL, anti-MICA/B, anti-NKG2D, anti-TRAIL or isotype control mAbs for 45 min, and then cocultured in 24-well plates. After 12 h incubation, viable cells were counted following staining with crystal violet.

FIGS. 12A-12C show a requirement for TRAIL-mediated tumor apoptosis. (A) OKT3-activated BTIL31 cells could kill both autologous and other tumor cells. BC20, BC29, BC31 or 1363mel cells were cocultured with same number of OKT3-activated or inactivated BTIL31-C1 cells in 96-well plates. After 12 h incubation, viable cells were counted after staining with crystal (B) Autologous BC31 tumor cells or OKT3 treatment on BTIL31 cells induced the TRAIL expression. BTIL31 cells were cocultured with BC29 and BC31 cells or cultured in the OKT3 pre-coated 24-well plates for 3 hrs, and TRAIL expression on BTIL31 cells were analyzed by FACS. (C) Expression of TRAIL receptors (DR4 and DR5) on tumor cells. All breast and melanoma cells expressed TRAIL receptor-2 (DR5), but not DR4 molecules. By contrast, neither T cells nor DCs expressed DR4 and DR5 molecules.

FIGS. 13A-13C demonstrate reversal of the suppressive function of γδ 1 Treg cells by TLR8 signaling. (A). Reversal of the suppressive function of γδ 1 T cells by TLR8 ligands, but not by ligands for other TLRs. naïve CD4⁺ T cells were cultured with BTIL31 cells at the ratio of 10:1 in the U bottom 96-well plates pre-coated with OKT3 in the presence of different TLR ligands: LPS, CpG-A, CpG-B, Imiquimod-R837, loxoribine, poly (I: C), ssRNA40/LyoVec, RNA33/LyoVec, pam3CSK4, flagellin and Poly-G3. In addition, naïve CD4⁺ T cells were cultured in the presence of supernatants derived from BTIL31 cells pre-treated with or without the indicated TLR ligands. The proliferation of naïve CD4⁺ T cells was determined by [³H]-thymidine incorporation assay described herein. Results are one representative data of three independent experiments. (B) Restoration of CFSE-naïve CD4⁺ T cell division by Poly-G3 oligonucleotides. CFSE-labeled Naïve CD4⁺ T cells cocultured with BTIL31 cells or its clones in the presence or absence of Poly-G3 in OKT3-coated 24-well plates. After 3 days of culture, cells were harvested and analyzed for divisions by FACS gated on the CFSE-labeled cells. CFSE-labeled Naïve CD4⁺ T cells alone served as a control. (C) Poly-G treatment had no effect on TRAIL-mediated killing activity. BTIL31 cells were cultured in the presence or absence of Poly-G3 for 2 days, and then tested the ability of Poly-G3-treated and untreated BTIL31 cells to kill BC31 autologous tumor cells.

### DETAILED DESCRIPTION OF THE INVENTION

### Definitions

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms. Some embodiments of the invention may consist of or consist essentially of one or more elements, method steps, and/or methods of the invention. It is contemplated that any method or composition described herein can be implemented with respect to any other method or composition described herein.

Unless defined otherwise, technical and scientific terms used herein have the same meaning as commonly understood by one of ordinary skill in the art to which this invention belongs. For purposes of the present invention, the following terms are defined below.

“An effective amount” is a concentration of composition, such as an oligonucleotide, for example, in a Treg cell's environment capable of inhibiting the Treg cell's immunosuppressive activity. The term “therapeutically effective amount” as used herein refers to an amount that results in an improvement or remediation of at least one symptom of a medical condition, such as cancer or infectious disease, for example.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.” Preferably, in nucleic acids comprising natural organic bases, the bases are unmethylated. Nucleic acid molecules can be obtained from existing nucleic acid sources but are preferably synthetic. The nucleic acids may comprise one or more of nuclease-resistant inter-residue backbone linkage and/or nuclease-sensitive inter-residue backbone linkage.

The term “library” includes searchable populations of molecules of a particular type. In one embodiment, the library is comprised of samples or test fractions (such as mixtures of small molecules or isolated small molecules, for example) that are capable of being screened for activity. For example, the samples could be added to wells in a manner suitable for high throughput screening assays. In a further embodiment, the library could be screened for binding compounds by contacting the library with a target of interest, e.g., a live cell, a protein or a nucleic acid. The type of molecule may be any type, but in specific embodiments, the molecule is an oligonucleotide, a small molecule, a peptide, a polypeptide, or a polynucleotide, for example.

“Type D CpG oligonucleotides” are known in the art, such as disclosed by U.S. Pat. No. 6,977,245 which is herein incorporated by reference in its entirety. Type D CpG oligonucleotides generally contain a CpG dinucleotide sequence and a stretch of 4 or more contiguous guanine residues. Type D CpG oligonucleotides are generally between 18 and 30 nucleotides in length, and may contain one or more of the following sequence content: 5′-X₁X₂TGCATCGATGCAGGGGGG-3′; (SEQ ID NO:10); 5′-X₁X₂TGCACCGGTGCAGGGGGG-3′; (SEQ ID NO:11); 5′-X₁X₂TGCGTCGACGCAGGGGGG-3′; (SEQ ID NO:12); 5′-X₁X₂TGCGCCGGCGCAGGGGGG-3′; (SEQ ID NO:13); 5′-GGTGCATCGATGCAGGGGGG-3′; (SEQ ID NO:14); 5′-GGTGCGTCGACGCAGGGGGG-3′; (SEQ ID NO: 15); 5′-GGTGCACCGGTGCAGGGGGG-3′; (SEQ ID NO: 16); or 5′-GGTGCATCGATGCAGGGGG-3′; (SEQ ID NO:17), where X may be any nucleobase or none.

A “non CpG containing recombinant DNA” is a recombinant DNA that does not contain a CpG dinucleotide sequence.

A “nuclease-resistant inter-residue backbone linkage” is a chemical linkage between organic bases in a nucleic acid that is more resistant to in vivo nuclease degradation as compared to naturally occurring phosphodiester linkages. The exemplary chemical linkage is a phosphorothioate (i.e., at least one of the phosphate oxygens of the nucleic acid molecule is replaced by sulfur) or phosphorodithioate modified nucleic acid molecules, for example. Other stabilized nucleic acid molecules include but are not limited to the following: nonionic DNA analogs, such as alkyl- and aryl-phosphonates, phosphodiester and alkylphosphotriesters, in which the charged oxygen moiety is alkylated. Nucleic acid molecules that comprise a diol, such as tetraethyleneglycol or hexaethyleneglycol, at either or both termini have also been shown to be substantially resistant to nuclease degradation.

A “nuclease-sensitive inter-residue backbone linkage” is a phosphodiester linkage between organic bases in a nucleic acid or alternative known in the art that degrades in vivo from nuclease activity at least at the same rate as a phosphodiester linkage.

An “immunogenic composition” is any composition capable of eliciting an immune response in a subject upon administration. The term “vaccine” as used herein is defined as material used to provoke an immune response (e.g., the production of antibodies) on administration of the materials and thus conferring immunity upon cleavage. Thus, a vaccine is an antigenic and/or immunogenic composition.

“Regulatory T cells” (Treg cells) are a functionally defined subset of T lymphocytes that function in vivo to control immunological reactivity to self antigens. This function is manifested by Treg cells' ability to suppress the activation of naïve immune effector cells (CD4⁺ and CD8⁺, for example), such as CD8⁺CD25⁻ T cells or γδ T cells, for example. At least two major classes of Treg cells are the thymically-derived natural Treg cells and antigen-induced Treg cells. Naturally occurring Treg cells mediate immunotolerance of self-antigens, and their dysregulation may play a role in autoimmune diseases. Antigen-induced Treg cells are induced by peripheral antigen stimulation. This subcategory of Treg cells is found among tumor infiltrating lymphocytes and mediates tolerance of tumor antigens. While Treg cell activation can be antigen-specific, Treg immunosuppression is not. Thus, Treg activity creates a globally suppressive immunological state. Treg cells may be characterized as a subpopulation of CD8⁺ T-cells expressing the IL-2 receptor CD25, although in alternative embodiments CD25 may not be expressed by the specific Treg cells, such as under some conditions, for example. Other molecular markers strongly associated with Treg cells are the transcription factor, FOXP3, and glucocorticoid-induced tumor necrosis factor receptor family-related gene (GITR, also known as TNFRSF18), for example. However, a skilled artisan recognizes that the best way to define Treg cells is to functionally determine their ability to inhibit the proliferation of naïve T cell proliferation as well as secretion of cytokines such as IL-2 by effector T cells.

“Cytokines” are small secreted proteins that mediate and regulate immunity, inflammation, and hematopoiesis. They must be produced de novo in response to an immune stimulus. They generally (although not always) act over short distances and short time spans and at very low concentration. They act by binding to specific membrane receptors, which then signal the cell via second messengers, often tyrosine kinases, to alter its behavior. Responses to cytokines include increasing or decreasing expression of membrane proteins (including cytokine receptors), proliferation, and secretion of effector molecules. Cytokine is a general name; other names include lymphokine (cytokines made by lymphocytes), monokine (cytokines made by monocytes), chemokine (cytokines with chemotactic activities), and interleukin (cytokines made by one leukocyte and acting on other leukocytes). Cytokines may act on the cells that secrete them (autocrine action), on nearby cells (paracrine action), or in some instances on distant cells (endocrine action). Cytokines are made by many cell populations, but the predominant producers are helper T cells (Th) and macrophages. The largest group of cytokines stimulates immune cell proliferation and differentiation. This group includes Interleukin 1 (IL-1), which activates T cells; IL-2, which stimulates proliferation of antigen-activated T and B cells; IL-4, IL-5, and IL-6, which stimulate proliferation and differentiation of B cells; Interferon gamma (IFNγ), which activates macrophages; and IL-3, IL-7 and Granulocyte Monocyte Colony-Stimulating Factor (GM-CSF), which stimulate hematopoiesis.

“Subject” or “individual” is an organism being given a composition, such as a nucleic acid, for example, including an oligonucleotide, according to the methods disclosed herein. In specific aspects, a subject or individual expresses a functional TLR8 on the subject's Treg cells. In further specific aspects, a subject or individual is a mammal other than mice (which do not express a functional TLR8), more preferably human.

The practice of the present invention will employ, unless otherwise indicated, conventional techniques of molecular biology, microbiology, recombinant DNA, and so forth which are within the skill of the art. Such techniques are explained fully in the literature. See e.g., Sambrook, Fritsch, and Maniatis, MOLECULAR CLONING: A LABORATORY MANUAL, Second Edition (1989), OLIGONUCLEOTIDE SYNTHESIS (M. J. Gait Ed., 1984), ANIMAL CELL CULTURE (R. I. Freshney, Ed., 1987), the series METHODS IN ENZYMOLOGY (Academic Press, Inc.); GENE TRANSFER VECTORS FOR MAMMALIAN CELLS (J. M. Miller and M. P. Calos eds. 1987), HANDBOOK OF EXPERIMENTAL IMMUNOLOGY, (D. M. Weir and C. C. Blackwell, Eds.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987), CURRENT PROTOCOLS IN IMMUNOLOGY (J. E. Coligan, A. M. Kruisbeek, D. H. Margulies, E. M. Shevach and W. Strober, eds., 1991); ANNUAL REVIEW OF IMMUNOLOGY; as well as monographs in journals such as ADVANCES IN IMMUNOLOGY.

U.S. Provisional Application Ser. No. 60/660,028, filed Mar. 9, 2005, and PCT Patent Application PCT/US06/08379, filed Mar. 9, 2006, are both incorporated by reference herein in their entirety.

### EMBODIMENTS OF THE PRESENT INVENTION

The present invention concerns a novel class of immunologically active oligonucleotides and methods to employ them for a therapeutic purpose. These immunologically active oligonucleotides, in specific aspects of the invention, act through Toll-Like Receptor 8 (TLR8) to activate the TLR8-IRKA4-MyD88 signal transduction pathway in regulatory T cells (Treg cells), for example. This signaling downregulates Treg cell activity, leading to a derepression of immunological activity.

Another embodiment of the invention relates to methods for inhibiting the immunosuppressive capacity of Treg cells utilizing this new class of immunologically active oligonucleotides. In particular embodiments of the invention, the immunosuppressive activity of Treg cells against naïve CD8⁺ T-cells or γδ T cells is downregulated by an effective amount of a guanine-comprising oligonucleotide. In a particular embodiment, Treg cell activity is down regulated in vitro with the effective amount determined by a titration series of oligonucleotide dosages, for example. In certain aspects of the invention, this Treg suppression method is effective with antigen-specific Treg cells from tumors and thymically-derived circulating Treg cells. Therefore, this method of suppressing Treg cell activity may be applied effectively in a wide variety of contexts, such as in subjects with an infectious disease or cancer, for example.

Regulatory T (Treg) cells play an important role in the maintenance of immunological self-tolerance by suppressing immune responses, thus preventing autoimmune diseases. However, such cells may also have detrimental effects on antitumor immunity. While elevated proportions of CD4⁺ CD25⁺ Treg cells have been demonstrated in several types of cancers, very little is known about the prevalence and subsets of Treg cells in prostate cancer. High percentages of CD4⁺ CD25⁺ T cells are present in the majority (70%) of prostate tumor-infiltrating T lymphocytes (PTILs), for example. Remarkably, at least both CD4⁺ and CD8⁺ T cell subpopulations possessed potent suppressive activity. T cell cloning and FACS analyses demonstrated that both CD4⁺ CD25⁺ and CD8⁺ CD25⁺ Treg cell clones derived from a bulk PTIL line expressed FoxP3 and suppressed naïve T cell proliferation, mainly through a cell contact-dependent mechanism. The suppressive function of Treg cells could be reversed by human Toll-like receptor 8 (TLR8) signaling, regardless of the subset of Treg cells and the suppressive mechanism involved, indicating that the manipulation of Treg cell function by TLR8 ligands improves the efficacy of immunotherapy for cancer patients, and particularly prostate cancer patients, in certain embodiments of the invention.

In other aspects of the invention, γδ⁺ TCR T cells play important roles in innate immunity against infectious pathogens and in surveillance of stressed or damaged tissues, inflammation, wound repair, and malignancy. However, their regulatory role in controlling the function T cells and dendritic cells (DCs) remains largely unknown. The inventors demonstrate herein that there is a dominant γδ1 T cell population in tumor-infiltrating lymphocytes (TILs) derived from breast and prostate cancers. These tumor-specific γδ1 T cells not only suppressed naïve T cell proliferation and IL-2 release from CD4⁺ and CD8⁺ effector cells, but also blocked the maturation and function of dendritic cells (DCs), indicating that they function as γδ regulatory T (Treg) cells. Although tumor-specific γδ1 Treg cells were capable of killing autologous tumor cells through a TRAIL-dependent pathway, they inhibited rather than killed T cells and DCs. The inventors further show that Toll-like receptor (TLR) 8 ligands, but not ligands for other TLRs, specifically reversed γδ1 Treg cell suppressive function, but their TRAIL-mediated killing activity was unchanged. These results provide new insights into tumor-specific γδ1 T cells and their distinct regulatory mechanisms for immune suppression and tumor immunity.

Use of the Invention for Medical Conditions

In certain embodiments, the present invention relates to methods and compositions for inhibiting at least partially an immune response related to CD8⁺ or γδ T cells. In certain aspects, such asn inhibition will increase the efficacy of a treatment for a medical condition. The immune response may be related to any medical condition of an individual, such as a mammal, including humans, dogs, cats, horses, pigs, cows, and so forth. In specific embodiments, the immune response that the invention concerns includes cancer, for example. Examples of types of cancer to which the present invention is relevant includes but is not limited to breast, prostate, melanoma, brain, colon, lung, ovarian, testicular, cervical, spleen, kidney, pancreatic, gall bladder, thyroid, bone, stomach, esophageal, liver, and so forth.

In other specific embodiments, the immune response concerns infectious disease. The infectious disease may be of any type, so long as the disease evokes an immune response. Exemplary infectious diseases include HIV, flu, cold, tuberculosis, cholera, anthrax, meningitis, brucellosis, dengue fever, diptheria, Ebola, encephalitis, Epstein-Barr virus, scarlet fever, yellow fever, malaria, measles, gonorrhea, hantavirus, hepatitis, lyme disease, mad cow disease, Norwalk infection, pertussis, polio, pneumonia, rabies, respiratory syncitial viral infection, Ricketts, ringworm, rotavirus, rubella, Rocky Mountain spotted fever, chicken pox, SARS infection, scabies, smallpox, shingles, St. Louis encephalitis, sleeping sickness, syphilis, tetanus, thrush, toxic shock syndrome, trichinosis, typhoid fever, ulcers, varicella, venereal disease, West Nile, and whooping cough, for example. The infectious disease may be bacterial or viral. The bacterial infection may be from the exemplary genera of Escherichia, Campylobacter; Candida, Clostridia, Cholera, Streptococcus, Staphylococcus, Helicobacter, Leishmania, Mycobacterium, Salmonella, Shigella, Treponema, Trypanosoma, and Vibrio, for example.

In particular aspects of the invention, a method and/or composition of the invention is performed or delivered to an individual that has a medical condition, such as cancer and/or infectious disease, for example. The methods and compositions of the present invention may be employed as a therapeutic composition, and in particular aspects, the compositions of the method are employed as a vaccine to prevent or suppress immunosuppresion by particular T cells in response to the cancer or infectious disease.

Compositions of the Invention

In certain aspects of the invention, there are compositions suitable for inhibiting the immunosuppressive activity of particular T cells, including, for example, CD8⁺ cells and/or γδ T cells. The composition may be of any suitable kind so long as they inhibit immunosuppressive activity of a particular T cell, but in particular aspects the composition comprises recombinant DNA. In specific embodiments, the compositions comprise oligonucleotides, and in further specific embodiments, the oligonucleotides are immunologically active oligonucleotides that provide an effect on an immune response, such as reducing the immune response, for example. Such a reduction in the immune response will improve the efficacy of at least one therapy for the medical condition of concern.

In certain embodiments of the invention, the compositions include one or more oligonucleotides. At least one of the oligonucleotide may comprise a guanosine and a partially stabilized or nuclease-resistant inter-residue backbone. In particular embodiments, the immunologically active oligonucleotides do not depend on having a CpG dinucleotide sequence. It is preferred that the oligonucleotide be a deoxyribonucleic acid for stability reasons, but other embodiments may alternatively include ribonucleic acids. The preferred length of the oligonucleotides is from about 4 to about 15 nucleotides, more preferably about 5 to about 10 nucleotides. In embodiments with partially-stabilized backbones, it is preferred to have a nuclease-resistant inter-residue linkage between a guanosine and an adjacent residue, for example. A representative group of oligonucleotides is shown as follows (wherein * indicates a nuclease resistant inter-residue backbone linkage): CpG-NG: A*AAAGACGATCG TCA *A*A*A*A*A (SEQ ID NO:5); Poly-G10: G*GGGG*G*G*G*G*G (SEQ ID NO:6); Poly-A10: A*AAAA*A*A*A*A*A (SEQ ID NO:7); Poly-T10:T*TTTT*T*T*T*T*T (SEQ ID NO:8); Poly-C10: C*CCCC*C*C*C*C*C (SEQ ID NO:9); Poly-G7: G*G*G*G*G*G*G; Poly-G5: G*G*G*G*G; Poly-G4: A G*G*G*G; Poly-G3: AG*G*GA; Poly-G2: AAG*GA; A4G1:CC*G*CC; T4G1: TT:G:TT; and G5: GGGGG, for example.

In particular aspects, the oligonucleotide compositions of the invention exclude CpG-A or Type D CpG oligonucleotides already known in the art. In other aspects, the oligonucleotide may be any oligonucleotide comprising one or more modified guanosine nucleotides, such as guanosine-diphosphoglucose or guanosine 5′-diphospho-D-Mannose, for example.

Methods to Screen for Compositions for their Ability to Reverse the Suppressive Function of Treg cells

Another embodiment of the invention relates to a new method for identifying compounds that inhibit the immunosuppressive capacity of CD8⁺ or γδ Treg cells.

### GENERAL EMBODIMENTS

In a certain embodiment, the method includes a comparison of cellular growth and/or division rates of parallel samples of naïve CD8⁺ or γδ T cells. Naïve cells, for example CD8⁺ or γδ T cells, exposed to uninhibited Treg cells are compared to 1) respective exemplary control naïve CD8⁺ or γδ T cells; and 2) respective naïve CD8⁺ or γδ T cells exposed to Treg cells treated with a candidate compound of interest. The reversal of Treg suppression is measured by the relative growths rates of the variously treated respective naïve CD8⁺ or γδ T cells. In a particular embodiment, the method for identifying compounds is used to screen a library or collection of candidate compounds. Such libraries are well known in the art and widely available (e.g., the NIH Molecular Libraries Small Molecule Repository). Lead compounds identified by a library screen can subsequently be modified to derive pharmaceutically acceptable compounds for reversing immunosuppression by Treg cells, in specific embodiments of the invention. In a preferred embodiment, the method for identifying compounds is semi- or fully automated using robotic systems and other devices well known in the art for high throughput library screening of cell based assays. (See, e.g., U.S. Pat. No. 6,400,487 Method and apparatus for screening chemical compounds).

In another screening embodiment, a plate comprising anti-CD3 antibodies but without antigen presenting cells is provided, and naïve T cells are presented thereto. After an appropriate period of time, such as about 56 hours, H³-thymidine is provided, and after another appropriate period of time, such as about 16 hours, proliferation is assessed (as a positive control, for example). Treg cells and naïve cells are then provided to the plates, and after an appropriate period of time, such as about 56 hours, H³-thymidine is provided; following another appropriate period of time, such as about 16 hours, proliferation is again assessed, and there should be no proliferation, as a screening control. Candidate TLR8 ligands or other compounds that can reverse Treg cell function are provided with naïve cells and Treg cells. Following an appropriate period of time, such as about 56 hours, H³-thymidine is provided, and after another appropriate period of time, such as about 16 hours, proliferation is assessed. If the candidate compound is indeed a TLR8 ligand or other compound that can reverse Treg cell function, then there is proliferation.

### Specific Embodiments

Naïve CD4⁺ T cells were purified from PBMCs by using microbeads (Miltenyi Biotec). Naïve CD4⁺ T cells (10⁵/well) were cultured with regulatory T cells at a ratio of 10:1 in OKT3 (2 μg/ml)-coated, U bottomed 96-well plates containing the following TLR ligands. LPS (100 ng/ml), imiquimod (10 μg/ml), loxoribine (500 μM), poly (I:C) (25 μg/ml), ssRNA40/LyoVec (3 μg/ml), ssRNA33/LyoVec (3 μg/ml), pam3CSK4 (200 ng/ml) and flagellin (10 μg/ml), all purchased from Invivogene (San Diego, Calif.). CpG-A (3 μg/ml), CpG-B (3 μg/ml) and poly-G3 oligonucleotides (3 μg/ml) were synthesized by Integrated DNA Technologies (Coralville, Iowa). All experiments were performed at least more than once.

Pharmaceutical Preparations

Pharmaceutical compositions of the present invention comprise an effective amount of one or more Treg cell activity-suppressing agent (which may be any kind of molecule, but in specific embodiments is a TLR8 ligand, and in further specific embodiments is an oligonucleotide) and, optionally, an additional agent, dissolved or dispersed in at least one pharmaceutically acceptable carrier. The phrases “pharmaceutically or pharmacologically acceptable” refers to molecular entities and compositions that do not produce an adverse, allergic or other untoward reaction when administered to an animal, such as, for example, a human, as appropriate. The preparation of a pharmaceutical composition that comprises at least one composition (such as an oligonucleotide, for example) capable of suppressing Treg cell activity will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The composition may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The composition may be formulated in a free base, neutral or salt form, where appropriate. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include the composition of the invention, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the composition may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according tot he response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

Alimentary Compositions and Formulations

In preferred embodiments of the present invention, the composition is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally- administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Parenteral Compositions and Formulations

In further embodiments, composition may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

Miscellaneous Pharmaceutical Compositions and Formulations

In other preferred embodiments of the invention, the active compound may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725, 871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

Combination Treatments

In certain aspects of the invention, it may be desirable to combine compositions of the invention with other agents. The other agents may be effective in the treatment of any medical condition for which the compositions of the present invention provide therapy by suppressing an immune response thereto. In specific aspects of the invention, a composition of the invention reduces the immune suppression of Treg cells and facilitates therapy by another agent. The medical condition may be a hyperproliferative disease, such as cancer, and the other agent may be an anti-cancer agent. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the composition of the invention and the additional agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

In other embodiments, the medical condition is an infectious disease, and the other agent may be an anti-infectious disease agent, such as an antibiotic (including an anti-bacterial agent) or antiviral agent. An antibiotic or anti-viral agent is capable of negatively affecting the infectious disease in the subject, for example, by reducing the amount of the pathogen attributed to the infectious disease, by killing cells harboring the pathogen attributed to the infectious disease, by inhibiting proliferation of the pathogen attributed to the infectious disease, or a combination thereof. This process may involve contacting the cells with the composition of the invention and the additional agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Delivery of the inventive composition may precede, follow, or be in conjunction with the other agent, and when they are not delivered concomitantly, the treatments may range in intervals from minutes to weeks. In embodiments where the other agent and inventive composition are applied separately to an individual or a cell therefrom, one would generally ensure that a significant period of time did not expire between the time of each delivery, such that the additional agent and composition of the invention would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, such as, for example, wherein the composition of the invention is “A” and the additional agent, such as radio- or chemotherapy for cancer and an antibiotic or antiviral agent for an infectious disease, is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of the compositions of the present invention to a subject will follow general protocols for the administration of therapeutics, taking into account the toxicity, if any, of the composition. It is expected that the treatment cycles would be repeated as necessary. It also is contemplated that various standard therapies, as well as surgical intervention, may be applied in combination with the described hyperproliferative cell therapy.

Combinations for Cancer Treatment

Wherein the medical condition is cancer and the composition of the invention suppresses the suppression of antitumor immunity from a Treg cell, the following additional agents may be employed:

Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein tansferase inhibitors, transplatinum, 5-fluorouracil, vincristin, vinblastin and methotrexate, or any analog or derivative variant of the foregoing.

Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with a TLR8 ligand or oligonucleotide that inhibits the antitumor immunity of Treg cells. The general approach for combined therapy is discussed below. Generally, the tumor cell must bear some marker or tumor antigens that are amenable to targeting, i.e., is not present on the majority of other cells. Many tumor markers or tumor antigens such as NY-ESO-1, TRP-1, TRP-2, gp100 exist and any of these may be suitable for targeting in the context of the present invention.

Dendritic cells may be employed as at least part of the immunotherapy. For example, dendritic cells can be transduced with an expression vector that is engineered to express a tumor antigen. Alternatively, dendritic cells can be pulsed/treated with a tumor antigen peptide.

Gene Therapy

In yet another embodiment, the secondary treatment is a gene therapy in which a therapeutic polynucleotide is administered before, after, or at the same time as a TLR8 ligand or immunosuppressive oligonucleotide. Delivery of a vector encoding a full length or truncated polynucleotide in conjuction with a composition of the invention will have a combined anti-hyperproliferative effect on target tissues. A variety of proteins are encompassed within the invention, some of which include inducers of cellular proliferation, inhibitors of cellular proliferation, and regulators of programmed cell death, for example.

Other Agents including Cytokine and Antibody Therapy

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

Combinations for Infectious Disease Treatment

Wherein the medical condition is cancer and the composition of the invention suppresses the suppression of antitumor immunity from a Treg cell, an antibiotic or antiviral agent may be employed. Exemplary antibiotics include penicillin, erythromycin, streptomycin, amoxicillin, gentamycin, ampicillin, cephalexin, doxycycline, fluconazole, ganciclovir, isoniazid, metronidazole, nistatin, rifampin, ticarcillin, and vancomycin. Exemplary antiviral agents include amantadine, rimantadine, oseltamivir, zanamivir, foscarnet, NM283, or bavituximab.

In vivo Delivery of Compositions

It is known in the art that compositions of the invention, which may be referred to as immune response-suppressing oligonucleotides, can be administered to a subject in any suitable manner. In certain aspects, the delivery occurs in conjunction with a vaccine, as an adjuvant, to boost a subject's immune system to effect better response from the vaccine. Lipford GB et al., CpG-containing synthetic oligonucleotides promote B and cytotoxic T cell responses to protein antigen: a new class of vaccine adjuvants. Eur J Immunol 27:2340-2344 (Sep. 1997). Analogous to the related art compositions, the oligonucleotides disclosed herein can likewise be co-administered. This will result in suppressed Treg cell activity associated with the immune response to the vaccine and improved overall immunogenicity of the vaccine.

Additionally, a wide variety of administrative routes are known in the art for delivery of oligonucleotides in vivo. Direct injection or systemic infusion have long been successfully applied in vivo for many oligonucleotides. See, e.g., Iverson, P., et al., “Pharmacokinetics of an Antisense Phosphorothioate Oligodeoxynucleotide against reve from Human Immunodeficiency Virus Type 1 in the Adult male Rate Following Single Injections and Continuous Infusion”, Antisense Research and Development, (1994), 4:43-52; Mojcik, C., et al., “Administration of a Phosphorothioate Oligonucleotide Antisense Murine Endogenous Retroviral MCF env Causes Immune Effect in vivo in a Sequence-Specific Manner”, Clinical Immunology and Immunopathology, (1993), 67:2:130-136; Krieg, et al., Immunostimulatory nucleic acid molecules, U.S. Pat. No. 6,239,116 (Filed Oct. 30, 1997). The choice of administrative route will be determined in part by reference to the disease. Solid tumors, for example, may be directly injected by bolus or continuous infusion with a pump, for example. Systemic veinous infusion may be used for diffuse malignancies, such as hematological cancers, for example. Similarly, subjects with localized infectious diseases will be preferably be given local injections or infusion while subjects with systemic infections will be given veinous infusions of oligonucleotides, for example.

Pharmaceutical preparations of other immune-modulating oligonucleotides are in the advanced stages of human clinical trial. See, e.g., Pfizer Pharmaceuticals, Inc., “Randomized Trial of Paclitaxel/Carboplatin ⁺PF-3512676 Vs Paclitaxel/Carboplatin Alone in Patients With Advanced NSCLC”, NCT00254891 (Phase III Clinical Trial), (November 2005).

As with related art oligonucleotides, compositions including the novel oligonucleotides disclosed herein will also be viable. Such compositions may contain pharmaceutically acceptable buffers, carriers and/or excipients that are well known in the art. Such ingredients will be capable of being co-mingled with the oligonucleotides of the present invention, and with each other, in a manner such that there is no interaction that would substantially impair the desired pharmaceutical efficiency of the oligonucleotides. Pharmaceutical compositions will contain an effective amount of oligonucleotide for suppressing Treg cell activity to effect an enhanced immune response to a disease state such as cancer or infection.

The oligonucleotides may also be combined with a delivery vector. A delivery vector can be anything capable of delivering oligonucleotides in vivo. A preferred vector is a colloidal dispersion system. Colloidal dispersion systems include lipid-based systems such as oil-in-water emulsions, micelles, mixed micelles, and liposomes. A preferred colloidal system is liposome based. Liposomes are artificial membrane vessels which are useful as a delivery vector in vivo or in vitro. It has been shown that large unilamellar vessels (LUV), which range in size from 0.2-4.0 μm can encapsulate large macromolecules. RNA, DNA and intact virions can be encapsulated within the aqueous interior and be delivered to cells in a biologically active form (Fraley, et al., Trends Biochem. Sci., (1981) 6:77); Gregoriadis, G. in Trends in Biotechnology, (1985) 3:235-241. Liposomes may be targeted to a particular tissue by coupling the liposome to a specific ligand such as a monoclonal antibody, sugar, glycolipid, or protein. Ligands which may be useful for targeting a liposome to an immune cell include, but are not limited to: intact or fragments of molecules which interact with immune cell specific receptors and molecules, such as antibodies, which interact with the cell surface markers of immune cells (e.g. CD25). Such ligands may easily be identified by binding assays well known to those of skill in the art. Liposomes are commercially available from Gibco BRL, for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB).

Parallels To CpG Oligonucleotide-Mediated Immunostimulatory Therapy

Krieg, et al., Immunostimulatory nucleic acid molecules, U.S. Pat. No. 6,239,116 (Filed Oct. 30, 1997) discloses a broad heterogeneous class of oligonucleotides sharing a common CpG sequence content. These oligonucleotides stimulate the immune system via Toll-Like Receptor 9 (TLR9), a molecular relative of Toll-like Receptor 8 (TLR8). In vivo, immune modulation using these CpG oligonucleotides functions via the same mechanisms dissected in vitro. The first step in CpG oligonucleotide immune stimulation is engaging TLR9 to induce intracellular signaling in dendritic cells, among others. Delivery and functional efficacy has been demonstrated in human clinical trials. See, e.g., NCT00254891 (Phase III Clinical Trial, November 2005). Delivery of the oligonucleotides of this specification will be similarly effected as described above and as with CpG oligonucleotides. The oligonucleotides of this specification will, analogous to CpG oligonucleotides and TLR9, engage TLR8 in vivo and thereby down regulate Treg suppressive activity.

However, there are significant differences between the approach of the present invention and and the Krieg TLR9 technology. Krieg's stimulation of TLR9 results in the up-regulation of dendritic cell signaling, etc. However, the present invention is not immunostimulatory in a direct sense, but rather acts to suppress the activity of several classes of T-regulatory cells. The present invention suppresses, not stimulates, a normal T-regulatory cell activity. The overall result is that antigen-specific immunity is enhanced because T-regulatory cell activity is suppressed. Therefore, although there are parallels between the Krieg approach and the present invention, there are also very important differences.

Parallels To Guanine Analog Pharmaceuticals

Imiquimod, a synthetic ligand for human TLR7 and 8, showed partial reversal of the suppressive function of antigen-specific Treg cells, but little or no effect on naturally occurring CD4⁺ CD25⁺ Treg cells. Resiquimod is a related compound with more potent pharmacological activity. Hengge UR, Ruzicka T., Topical immunomodulation in dermatology: potential of toll-like receptor agonists. Dermatol Surg. 2004 August; 30(8):1101-12. These agents act in part through TLR8. Mclnturff J E, Modlin R L, Kim J., The role of toll-like receptors in the pathogenesis and treatment of dermatological disease. J Invest Dermatol. 2005 July; 125(1):1-8; McCluskie M J, et al., Treatment of intravaginal HSV-2 infection in mice: A comparison of CpG oligodeoxynucleotides and resiquimod (R-848). Antiviral Res. 2006 February; 69(2):77-85. Topical compositions of imidazoquinolines (Imiquimod and Resiquimod) have been successfully applied to combat dermatological viral infections, such as human papillomavirus, herpes simplex virus, and mollusca, and skin cancer. Naylor M., Imiquimod and superficial skin cancers. J Drugs Dermatol. 2005 September-October; 4(5):598-606; Chang Y C, et al., Current and potential uses of imiquimod. South Med J. 2005 September; 98(9):914-20. While these compounds have demonstrated the efficacy of immunomodulation via TLR8 to effect positive clinical outcomes, imidazoquinolines are thus far only suitable for external administration. The oligonucleotides and methods of their use disclosed by this Specification will permit systemic and localized internal administrations to effect similar TLR8 mediate results in a much broader set of disease conditions.

Previously Established Efficacy of Treg Suppression in Cancer and Infectious Diseases

Several infectious diseases become persistent due to suboptimal immune response. It is hypothesized that this may be a homeostatic mechanism to keep some pathogens in check while preventing collateral systemic damage from a more aggressive immune response. Extensive experimentation in a variety of infectious disease models has demonstrated that neutralizing Treg cells can boost immune response and improve outcomes.

Leishmaniasis major can persist as a localized homeostatic infectious state after a primary infection. This static infected state is mediated by Treg cell activity that prevents complete elimination of the infection. This may represent a favored symbiotic status, because persistent localized infection results in much improved immune response to a re-infection distal from the static infection site. Treg suppression can be mediated by anti-CD25 depletion of CD25⁺ cells. This effectively depletes the majority of Treg cells and results in a sterilizing immune response that eliminates a persistent infection. Belkaid Y, CD4⁺CD25⁺ regulatory T cells control Leishmania major persistence and immunity. Nature. 2002 Dec. 5; 420(6915):502-7. This result is proof of principle that suppressing Treg cell activity in vivo can effect immunostimulation with clinically relevant results.

The efficacy of Treg suppression has been demonstrated in a wide array of diseases such as the exemplary following: C. albicans (Montagnoli C, B7/CD28-dependent CD4⁺ CD25⁺ regulatory T cells are essential components of the memory-protective immunity to Candida albicans. J Immunol. 2002 Dec. 1; 169(11):6298-308.); Malaria (Hisaeda H, Escape of malaria parasites from host immunity requires CD4⁺ CD25⁺ regulatory T cells. Nat Med. 2004 January; 10(1):29-30.); Human Immunodeficiency Virus (Kinter Ala., CD25(⁺)CD4(⁺) regulatory T cells from the peripheral blood of asymptomatic HIV-infected individuals regulate CD4(+) and CD8(⁺) HIV-specific T cell immune responses in vitro and are associated with favorable clinical markers of disease status. J Exp Med. 2004 Aug. 2; 200(3):331-43; Aandahl E M, Human CD4⁺ CD25⁺ regulatory T cells control T-cell responses to human immunodeficiency virus and cytomegalovirus antigens. J Virol. 2004 Mar; 78(5):2454-9.).

Analogous Treg suppression results demonstrate efficacy against a variety of cancer types as well. Some of these results include the exemplary following: Leukemia, melanoma, plasmacytoma and mastocytoma (Shimizu J, Induction of tumor immunity by removing CD25⁺CD4⁺ T cells: a common basis between tumor immunity and autoimmunity. J Immunol. 1999 Nov 15; 163(10):5211-8.); Leukemia, myeloma, and sarcoma (Onizuka S, Tumor rejection by in vivo administration of anti-CD25 (interleukin-2 receptor alpha) monoclonal antibody. Cancer Res. 1999 Jul 1; 59(13):3128-33.); Melanoma (Sutmuller R P, Synergism of cytotoxic T lymphocyte-associated antigen 4 blockade and depletion of CD25(⁺) regulatory T cells in antitumor therapy reveals alternative pathways for suppression of autoreactive cytotoxic T lymphocyte responses. J Exp Med. 2001 Sep. 17; 194(6):823-32.).

While effective, these treatments involve immunodepletion with anti-CD25 or anti-GITR antibodies. This approach is less desirable because these molecular entities are also associated with non-Treg cell populations. Thus, delivery of the recombinant DNA described in this specification, as described above, will provide a less destructive and more attenuable alternative.

Kits of the Invention

Any of the compositions described herein may be comprised in a kit. In a non-limiting example, an immunosuppressive oligonucleotide may be comprised in a kit. The kits will thus comprise, in suitable container means, an immunosuppressive oligonucleotide of the present invention and, optionally, an additional agent.

The kits may comprise one or more suitably-aliquoted immunosuppressive oligonucleotide composition of the present invention, whether labeled or unlabeled. The components of the kits may be packaged either in aqueous media or in lyophilized form, for example. The container means of the kits will generally include at least one vial, test tube, flask, bottle, syringe or other container means, into which a component may be placed, and preferably, suitably aliquoted. Where there are more than one components in the kit, the kit also will generally comprise a second, third or other additional container into which the additional component(s) may be separately placed. However, various combinations of components may be comprised in a vial. The kits of the present invention also will typically include a means for containing the immunogenic oligonucleotide and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred. The composition may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

Irrespective of the number and/or type of containers, the kits of the invention may also comprise, and/or be packaged with, an instrument for assisting with the injection/administration and/or placement of the ultimate composition within the body of an animal. Such an instrument may be a syringe, pipette, forceps, and/or any such medically approved delivery vehicle.

The kit may further comprise a therapeutic agent, such as an anti-cancer agent or an antibiotic or antiviral agent of any kind, including the specific examples noted in section VIII above.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Regulatory T Cells in Prostate Cancer

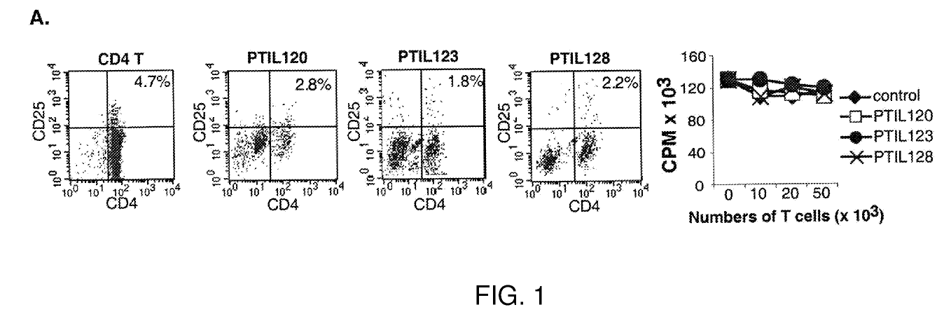

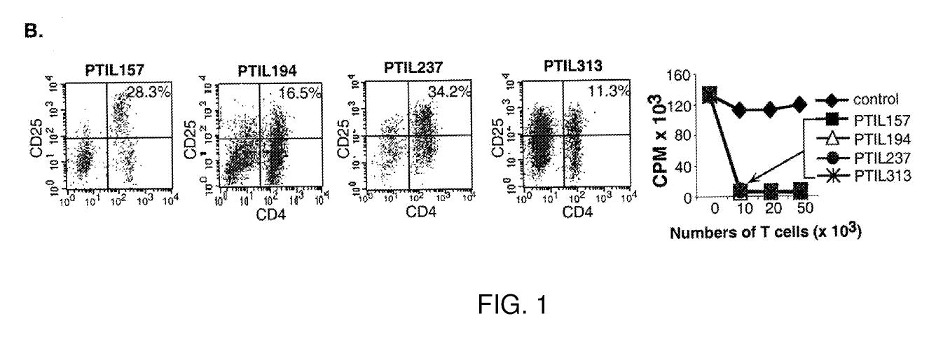

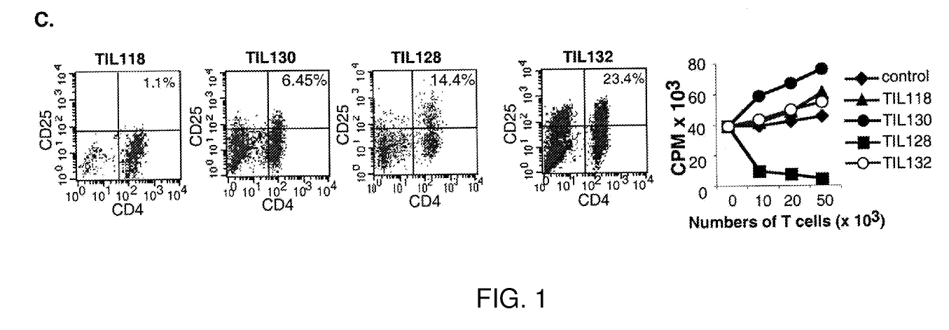

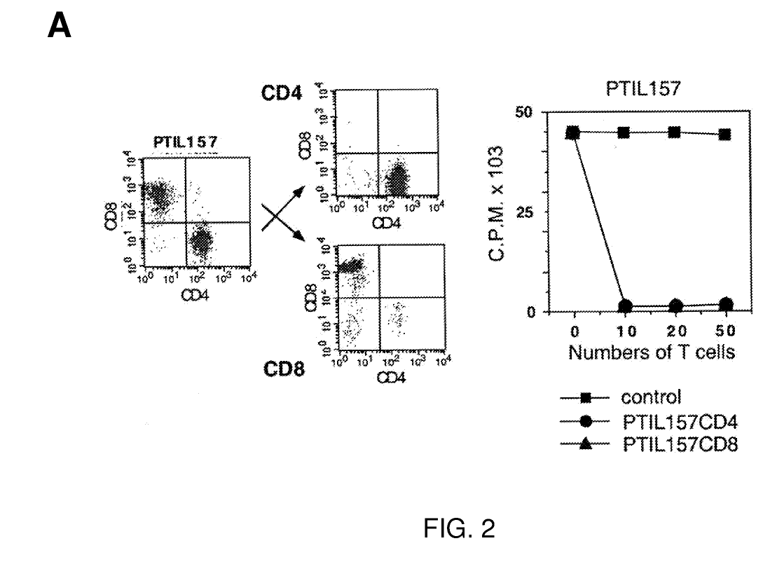

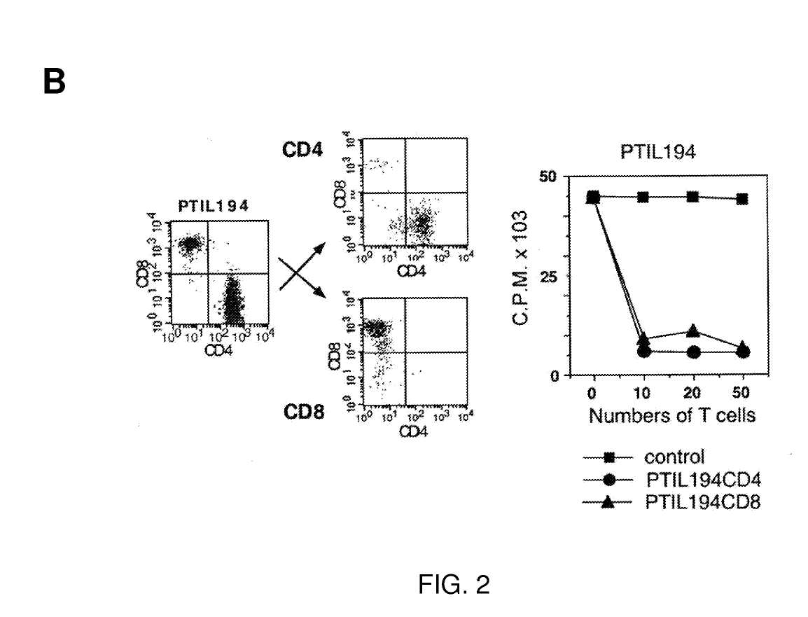

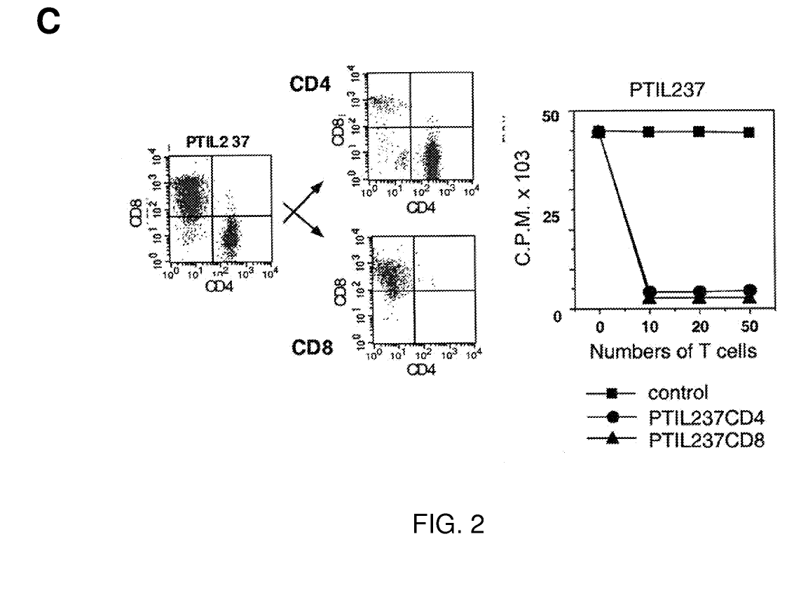

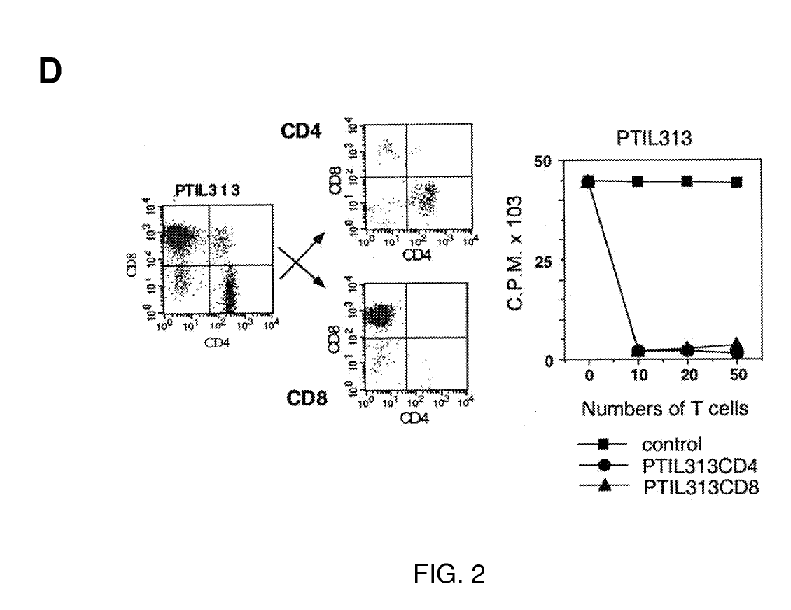

Increased percentages of CD4⁺ CD25⁺ Treg cells have been found in several types of cancers (Woo et al., 2001; Curiel et al., 2004), but very little is known about Treg cells in human prostate cancer. The inventors therefore established 52 TIL cell lines from 200 prostate tumor samples, maintained them in culture for at least 3-4 weeks to obtain enough number of cells for further analysis. FACS analysis of 22 TIL lines identified two discrete subsets based on the expression of CD4 and CD25 molecules. Six (28%) of 22 TILs contained less than 5% CD4⁺ CD25⁺ T cells in the total T-cell population, and did not produce a suppressive effect on naïve T cell proliferation, while the remaining 16 TILs (72%), including PTIL157, PTIL194, PTIL237 and PTIL313, contained elevated percentages (11-34%) of CD4⁺ CD25⁺ T cells in the total T-cell population, and showed a potent ability to suppress naïve T cell proliferation. Representative data are shown in FIGS. 1A, 1B.

Since melanoma is a relatively immunogenic human cancer and the associated TILs are relatively easy to grow in culture, the percentages of Treg cells from prostate tumors were compared with those from melanoma. Of 10 melanoma-derived TILs, 3 showed an increased proportion of CD4⁺ CD25⁺ T cells; the remaining melanoma-derived TILs contained low or normal percentages of CD4⁺ CD25⁺ T cells in the total T-cell population. In contrast to the suppressive activity of bulk prostate TILs with a high percentage of CD4⁺ CD25⁺ T cells, most melanoma TILs, regardless of percentage of CD4⁺ CD25⁺ T cells, did not have a suppressive effect on naïve T cell proliferation (FIG. 1C). These results suggest that the majority of prostate cancer-derived TILs, but only a small percentage of melanoma-derived TILs, contained elevated proportion of CD4⁺ CD25⁺ T cells and exhibited suppressive activity, which may explain why melanoma-derived T cells are relatively easy to grow and expand in vitro.

### Example 2

### Suppression of Naïve T Cell Proliferation by CD4⁺ and CD8⁺ Treg Cell Lines/Clones Derived from Exemplary Prostate Tumor-Derived Til Cell Lines

To determine the subsets of Treg cells responsible for the observed suppression of naïve T cell proliferation, 4 bulk TIL cell lines (PTIL157, PTIL194, PTIL237 and PTIL313) were selected for further analysis. CD4⁺ and CD8⁺ T-cell subpopulations were purified from bulk T cell lines with anti-CD4 or anti-CD8 antibody-coated magnetic beads and tested for their ability to inhibit the proliferation of naïve CD4⁺ T cells. As expected, CD4⁺ T-cell population showed a marked suppressive effect; however, CD8⁺ T populations isolated from 4 TILs were suppressive, indicating that the purified CD8⁺ T-cell population contained CD8⁺ Treg cells.

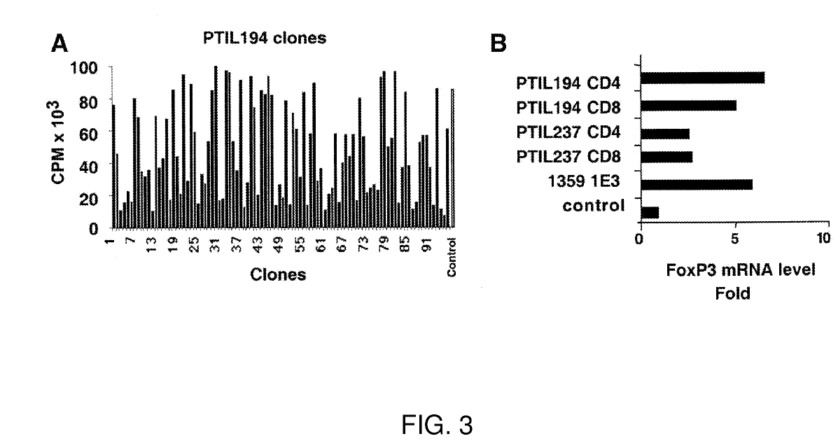

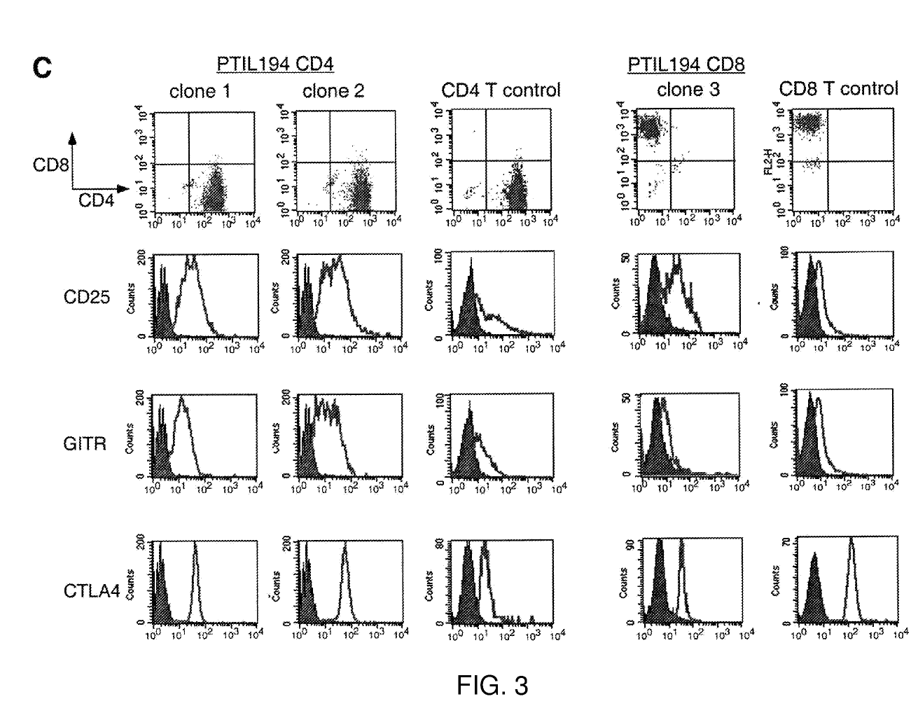

To demonstrate the co-existence of CD4⁺ and CD8⁺ Treg cells in prostate cancer-derived TILs, T cell clones were generated from PTIL194 by limiting dilution cloning. More than 100 T-cell clones were obtained and analyzed for their ability to inhibit naïve T cell proliferation in a functional assay. A representative set of data is shown in FIG. 3A. Among 94 T cell clones, 51 had strong suppressive activity, while 43 had little or no suppressive activity. To determine the cell phenotype of clones with suppressive activity, FACS analysis was performed and identified a mix of CD4⁺ and CD8⁺ clones. As shown in FIG. 3B, CD4⁺ T-cell clones with a suppressive function were positive for CD25 and GITR molecules, while nonsuppressive CD4⁺ effector cells were negative for these markers. Importantly, the suppressive CD8⁺ T cells (clone 3) were positive for CD25, but their expression of GITR did not differ from that of nonsuppressive CD8⁺ control cells.

In addition, real-time PCR was performed to evaluate the mRNA expression level of FoxP3 in CD4⁺ and CD8⁺ populations as well as Treg cell clones. PTIL194, PTIL237 and Treg cell clones expressed a 5-fold higher level of FoxP3 than did the control effector T cells. 1359 1E3 clone served as a positive control for FoxP3 expression in Treg cells. Bulk TIL lines and Treg cell clones secreted a large amount of IFN-γ, but little or no IL-2, IL-4 or IL-10 after stimulation with anti-CD3 antibody. Taken together, these results indicate that prostate tumor-derived TIL lines contain both CD4⁺ and CD8⁺ Treg cells, which expressed FoxP3 and suppressed the proliferation of naïve T cells.

### Example 3

### Suppressive Mechanisms of Prostate Tumor-Derived Treg Cells

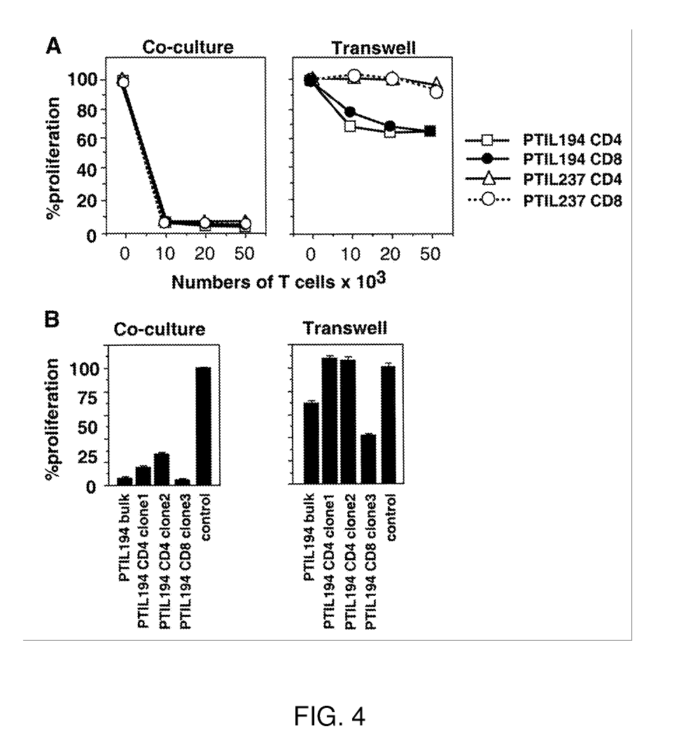

The suppressive mechanisms of prostate tumor-derived Treg cells were further characterized. Although both CD4⁺ and CD8⁺ T-cell populations from PTIL194 and PTIL237 inhibited naïve CD4 T cell proliferation in the co-culture assay condition, T cells from PTIL237 could not suppress naïve CD4 T-cell proliferation in a transwell system (FIG. 4A), suggesting that cell-cell contact is required for immune suppression by PTIL237. However, T cells from PTIL194 showed partial (20%) inhibition of naïve T cells in a transwell system (FIG. 4A), indicating that some Treg cells in PTIL194 inhibit naïve T cell proliferation through a cell-contact-dependent mechanism, while others suppress immune responses via soluble factors (IL-10 and/or TGF-β). However, the addition of anti-IL10, anti-TGF-β or both antibodies could not restore naïve T-cell proliferation. Indeed, two CD4⁺ Treg cell clones from PTIL194 could not inhibit naïve CD4 T cell proliferation in a transwell assay, while the CD8⁺ Treg cell clone could inhibit naïve T cell proliferation, even in a transwell system. These results indicate that although both soluble factor-dependent and cell contact-dependent suppressive mechanisms could be used by Treg cells derived from prostate tumor-derived T cells, the latter is perhaps the predominant mode, in certain aspects of the invention.

### Example 4

### Reversal Of CD4⁺ and CD8⁺ Treg Cell Function by TLR8 Ligands

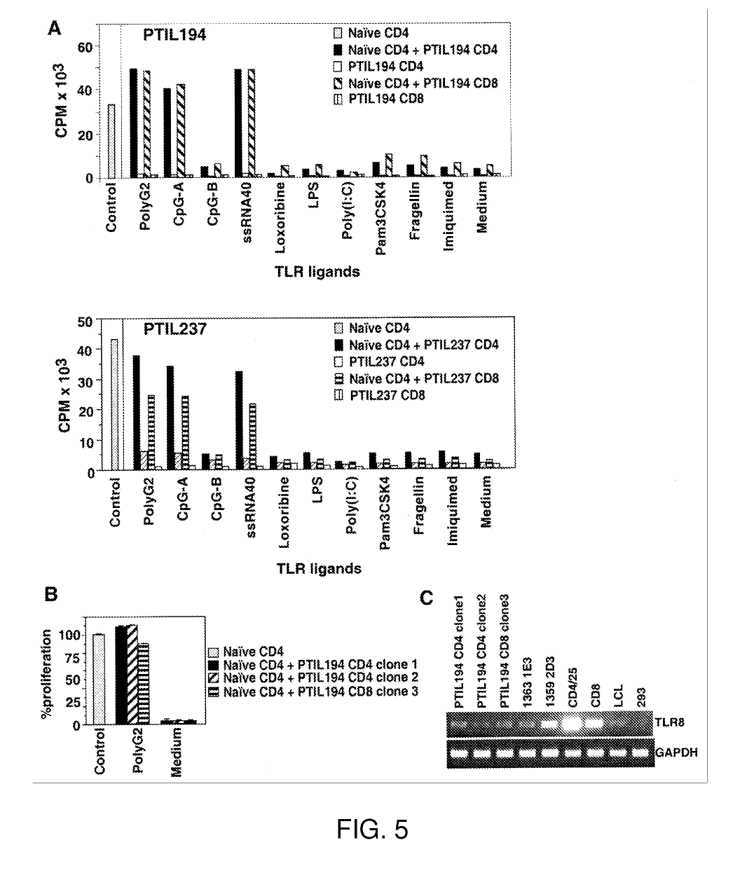

The inventors recently demonstrated that the suppressive function of CD4⁺ Treg cells can be reversed by TLR ligands (Peng et al., 2005), but it was not clear whether the same TLR8 signaling also applies to CD8⁺ CD25⁺ Treg cells. To test this possibility, the suppressive effects of CD4⁺ and CD8⁺ Treg cells were examined on naïve T cell proliferation in a functional assay, with or without TLR8 ligands (Poly-G2 and ssRNA40) or ligands for other TLRs. As expected, the suppressive activity of CD4⁺ Treg-containing cell populations and Treg cell clones was abolished by TLR8 ligand poly-G2 and ssRNA40, but was not affected by ligands for other TLRs (FIGS. 5A, 5B). Interestingly, similar reversal patterns were observed for CD8⁺ Treg-containing cell populations and clones, suggesting that TLR8 signaling controls the suppressive function of both CD4⁺ and CD8⁺ Treg cells, a notion supported by PCR analysis of TLR8 expression in CD4⁺ and CD8⁺ Treg cells (FIG. 5C), although there were variations of TLR8 expression in different Treg cell lines/clones. Thus, besides their similarities in phenotypic and suppressive mechanisms between CD4⁺ and CD8⁺ Treg cells, they also share a common pathway for their functional regulation through the TLR8 signaling.

### Example 5

### Significance of Suppression of Prostate Tumor-Derived Regulatory T Cells

Increasing evidence indicates that tumor-infiltrating immune cells play a major role in combating cancer and their activity correlates with disease prognosis and survival (Zhang et al., 2003; Sato et al., 2005). In the most cases, however, tumor-specific T cells ultimately fail to control tumor growth. Spontaneous tumor regression is relatively rare. If these tumor-specific T cells are expanded ex vivo and then adoptively transferred together with IL-2 into autologous patients, approximately 30% of the patients show objective tumor regression (Rosenberg, 2001). It was recently demonstrated that the clinical response rate could be improved as much as 50% if patients scheduled to receive adoptive T-cell therapy were first conditioned with cyclophosphamide for 2 days to remove whole-body lymphocytes (Dudley et al., 2002). These results indicate that tumor-specific T cells may be suppressed by Treg cells in the tumor microenvironment, and their removal by cyclophosphamide may enhance antitumor immunity.

Although elevated proportions of Treg cells have been reported in patients with other cancers (Woo et al., 2001; Curiel et al., 2004), the present invention is the first to analyze in detail the prevalence of different Treg cells subpopulations as well as their suppressive mechanisms in prostate cancer patients. Several lines of evidence suggest that prostate-derived TILs are distinct from those derived from other types of cancers. First, it was found that the majority (70%) of prostate tumor-derived TILs contained high percentages of CD4⁺ CD25⁺ Treg cells, which suppressed naïve T-cell proliferation. By contrast, less than 30% of TILs derived from melanoma had a high percentage of CD4⁺ CD25⁺ Treg cells. The second unique feature is that prostate tumor-derived CD8⁺ CD25⁺ Foxp3⁺ Treg cells suppressed immune responses. Thus, prostate tumor environment appears to contain both CD4⁺ and CD8⁺ Treg cells that can inhibit antitumor immunity, which may explain, at least in part, why prostate cancer is poorly immunogenic and why their associated T cells are generally difficult to grow in vitro. Using a transgenic mouse model of prostate tumor, Tien et al found an increased frequency and number of CD4⁺CD25⁺ T cells and an enhanced production of inhibitory cytokines during tumor progression (Tien et al., 2005).

In contrast to CD4⁺ Treg cells, much less is known about CD8⁺ Treg cells (Jiang and Chess, 2004). CD8⁺ Treg cells have been identified to mediate immune suppression in an antigen-dependent manner (Jiang and Chess, 2004; Sarantopoulos et al., 2004; Vlad et al., 2005). CD8⁺ Treg cells suppress antigen-activated CD4⁺ T cells in a TCR specific manner restricted by the MHC class Ib molecule, Qa-1 (Jiang and Chess, 2000; Hu et al., 2004). However, recent studies demonstrated that CD8⁺ CD122⁺ (IL-2/IL15 receptor β chain) Treg cells can prevent the development of abnormally activated T cell-mediated disease in CD 122-deficient mice (Rifa'l et al., 2004). CD8⁺ CD25⁺ Treg cells have recently been isolated from human peripheral blood mononuclear cells (PBMCs) and MHC class II-deficient mice (Cosmi et al., 2003; Jarvis et al., 2005; Bienvenu et al., 2005). These CD8⁺ Treg cells can suppress immune responses in an antigen nonspecific manner. In this invention, it was shown that prostate tumor-derived CD8⁺ Treg cells expressed CD25 and Foxp3 molecules (FIG. 3), which are shared by CD4⁺ Treg cells. Although both CD4⁺ and CD8⁺ Treg cells are positive for CTLA-4 and GITR molecules, there was no any appreciable difference in expression level of CTLA-4 between CD4⁺ Treg, CD8⁺ Treg and effector T cells, whereas the expression level of GITR expression in CD4⁺ Treg cells was higher than that in CD4⁺ effector cells (FIG. 3). Foxp3 has proved to be a relative specific marker for Treg cells and critical to the development of Treg cells (Hori et al., 2003; Fontenot et al., 2003). It should be noted that Foxp3 expression in mouse is restricted to CD4⁺ Treg cells, but little or no expression in CD8⁺ T cells and other cell population (Fontenot et al., 2005). In contrast, Foxp3 expression in human is not so restricted, and has been detected in many subsets of T cells (Morgan et al., 2005; Roncador et al., 2005; Ziegler, 2006; Gavin et al., 2006), although its expression level in Treg cells is higher than that in effector cells. Although it is well recognized that CD4⁺ Treg cells suppress immune responses through cell-contact dependent or soluble factor dependent mechanisms (Sakaguchi, 2004), both prostate tumor-derived CD4⁺ and CD8⁺ Treg cells inhibit naïve T cell proliferation mainly through a cell-contact dependent mechanism. It appears that both CD4⁺ and CD8⁺ Treg cells share some phenotypic markers and suppressive mechanisms.

These findings raise an intriguing question: what is the mechanism that allows CD4⁺ and CD8⁺ Treg cells to accumulate in the prostate tumor microenvironment. There may be sequential events occurred in the process. First, recent studies have linked chronic inflammation to cancer development and progression (Coussens and Werb, 2002; Greten et al., 2004; Karin and Greten, 2005). Suppressive cytokines such as IL-10 and TGF-β and chemokines such as CCL22 secreted by tumor cells or tumor infiltrating macrophages, myeloid suppressor cells and DCs not only recruit Treg cells to tumor sites, but also favor the conversion of nonsuppressive T cells into Treg cells with suppressive function (Curiel et al., 2004; Huang et al., 2006). This notion is supported by the findings of the inventors showing that prostate tumor-derived Treg cells express CCR4, a receptor for CCL22. Second, it is likely that tumor cells may actively recruit, activate and expand Treg cells by either directly or indirectly presenting antigenic peptides for their recognition. Indeed, previous studies demonstrated that tumor cells express tumor-specific antigens such as LAGE1 and ARTC1 and directly stimulate antigen-specific Treg cells (Wang et al., 2004; Wang et al., 2005). Since some prostate tumor-derived TILs had tumor-specific recognition, it is reasonable to believe that tumor antigens expressed by prostate tumor cells may play a critical role in the recruitment, activation and maintenance of Treg cells at tumor sites. In addition, it has been demonstrated that immunization of mice with serological identification of antigens by recombinant expression cloning (SEREX)-defined autoantigen DNA J-like 2 can induce the generation of Treg cells (Nishikawa et al., 2003). Finally, chronic and low-dose repetitive antigen stimulation can convert CD4⁺ naïve or effector T cells into Treg cells in animal models (Klein et al., 2003; Kretschmer et al., 2005). Thus, antigens expressed by tumor cells, soluble factors and cytokines/chemokines in tumor microenvironments may play a critical role in recruiting, expanding and maintaining Treg cells at tumor sites (Wang, 2006).

Regardless of how regulatory T cells accumulate near the tumor sites of prostate, the depletion or removal of CD4⁺ CD25⁺ regulatory T cells could be expected to improve antitumor immune responses. Indeed, the depletion of such Treg cells using anti-CD25 mAb enhanced antitumor immunity in vivo (Onizuka et al., 1999; Tien et al., 2005). Further testing of this concept in clinical trials using an IL-2/diphtheria toxin fusion protein (denileukin diftitox, or Ontak) indicated that the depletion of CD4⁺ CD25⁺ T cells by Ontak could enhance antitumor activity, although Ontak also depleted newly activated CD4⁺ CD25⁺ effector cells (Dannull et al., 2005). However, Another study by Attia et al showed that Ontak was ineffective in depleting CD4⁺ CD25⁺ T cells (Attia et al., 2005). Hence, even if this fusion protein is capable of depleting CD25⁺ T cells, its use may limit to pre-vaccination treatment. The inventors recently demonstrated that TLR8 signaling could reverse the suppressive function of naturally occurring Treg as well as tumor-specific Treg cells (Peng et al., 2005). Here the inventors show that TLR8 ligands not only reversed the suppressive function of CD4⁺ Treg cells, but also blocked CD8⁺ Treg cell suppressive function (FIG. 5), implying that CD4⁺ and CD8⁺ Treg cells may share a common suppressive mechanism, which can be reversed by triggering TLR8 signaling pathway. It is not clear why the ligands for other TLRs failed to modulate the suppressive function of either CD4⁺ or CD8⁺ Treg cells. One plausible explanation is that expression pattern of TLRs on Treg cells may partially account for the specificity of TLR8-mediated functional reversal of Treg cells since human Treg cells consistently express high level of TLR8, but little or no TLR7 and TLR9 (Peng et al., 2005). By contrast, CD4 effector or memory T cells express little or no TLR8, but a high level of TLR1, 2, 3, 4 and 5, and a low level of TLR7 and 9 mRNA (Caron et al., 2005). The conclusion of the inventors was further supported by a recent study showing that stimulation of human CD4⁺ Treg cells with TLR5 ligands did not reverse their suppressive function, but rather enhance their suppressive function and Foxp3 expression (Crellin et al., 2005). Taken together, triggering of TLR8 signaling by its ligands reverse the suppressive function of human CD4⁺ and CD8⁺ Treg cells, while ligation of other TLRs with their respective ligands enhance rather than reverse Treg cell suppressive function. Since TLR8 is not functional in mice (Jurk et al., 2002), the regulation of murine Treg cells through TLR signaling may differ from the functional control of human Treg cells. Indeed, it was recently reported that TLR2 promoted the proliferation of both murine CD4⁺ Treg and effector cells, and transiently abrogated the suppressive function of murine CD4⁺ Treg cells (Sutmuller et al., 2006; Liu et al., 2006). Regardless of these differences in the functional regulation of Treg cells between humans and mice, it may be possible to use TLR ligands to manipulate Treg cell suppressive function as well as effector T cells, thus shifting the balance between Treg and effector cells. This new strategy may prove useful in improving the efficacy of cancer vaccines against prostate and other cancers.

### Example 6

### Exemplary Materials and Methods for Examples 1-5

Generation of Tumor-Infiltrating T Cells and T-Cell Cloning

Prostate cancer tissues were minced into small pieces followed by digestion with triple enzymes mixture containing collagenase type IV, hyaronidase and deoxyribonuclease for 2 hours at room temperature. After digestion, the cells were washed twice in RPMI1640 and cultured in RPMI1640 containing 10% human serum supplemented with L-glutamine and 2-mercaptethanol and 1000 U/ml of IL-2 for the generation of T cells over 2-3 weeks. Experiments for human materials and tumor sample collection is conducted under the IRB protocol (H-9086) approved by Baylor College of Medicine IRB committee. T cell clones were generated from TILs by the limiting dilution cloning method, as previously reported (Wang et al., 2004). T cell clones were transferred to fresh 96-well plates and used in a functional assay to determine their ability to inhibit naïve T cell proliferation. Some T cell clones with suppressive activity were selected for further analyses. FACS analysis of CD25 and GITR

CD4⁺ and CD8⁺ T cell populations were purified with specific antibody-coated beads. The expression of CD25 and GITR on Treg cells was determined by FACS analysis after staining with specific antibodies (purchased from R&D Systems and BD Biosciences), as previously described (Wang et al., 2004; Wang et al., 2005).

Proliferation Assays and Transwell Experiments

CD4⁺ CD25-T cells (2×10⁵) purified from human PBMCs by antibody-coated beads (Dynal, Inc.) were cultured for 60 hours in U-bottomed 96-well plates containing 5×104 CD3-depleted APCs, 0.1 μg/ml anti-CD3 mAb, and different numbers of CD4⁺ regulatory or effector T cells. The proliferation of responder T cells was determined by the incorporation of [H³]thymidine for the last 16 hours of culture, as previously described (Wang et al., 2004; Wang et al., 2005). Cells were harvested and the radioactivity counted in a scintillation counter. All experiments were performed in triplicate. Transwell experiments were performed in 24-well plates with a pore size of 0.4 μm (Coming Costar, Cambridge, Mass.). 2×10⁵ the freshly purified naïve CD4⁺ T cells were cultured in the outer wells of 24-well plate in medium containing 0.1 μg/ml anti-CD3 antibody and 2×10⁵ APCs. Equal numbers of regulatory T cells or nonregulatory T cells were added into the inner wells in the same medium containing 0.5 μg/ml anti-CD3 antibody and 2×10⁵ APCs. After 56 hours of culture, the cells in the outer and inner wells were harvested separately and transferred to 96-well plates. [H³]thymidine was added, and the cells were cultured for an additional 16 hours before harvest for the radioactivity counting with a liquid scintillation counter.

PCR Analysis of FoxP3 and TLR8

Total RNA was extracted from 1×10⁷ T cells using Trizol reagent (Invitrogen, Inc. San Diego, Calif.). A SuperScript II RT kit (Invitrogen, Inc. San Diego, Calif.) was used to perform reverse transcription, in which 20 μl of the reverse transcription mixture, containing 2 μg of total RNA, was incubated at 42° C. for 1 hour. FoxP3 mRNA levels were quantified by real-time PCR using ABI/PRISM7000 sequence detection system (PE Applied Biosystems, Inc. Foster City, Calif.). The PCR reaction was performed with primers and an internal fluorescent TaqMan probe specific for FoxP3 or HPRT, all purchased from PE Applied Biosystems Inc. (Foster City, Calif.). FoxP3 mRNA levels in each sample were normalized with the relative quantity of HPRT. All samples were run in triplicate.

For analysis of TLR8, PCR analysis was performed with the TLR8 forward primer, 5′-TTTCCCACCTACCCTCTGGCTT-3′ (SEQ ID NO:1), and the reverse primer, 5′-TGCTCTGCATGAGG TTGTCGGATGA-3′ (SEQ ID NO:2). PCR amplification for glyceraldehyde-3-phosphate dehydrogenase (GAPDH) served as a PCR control (forward primer, 5′-CGAGATCCCTCCAAAATCAA (SEQ ID NO:3), and reverse primer, 5′-TGTGGTCATGAGTCCTTCCA; SEQ ID NO:4).

Toll-Like Receptor Ligands and Proliferation Assays

Naïve CD4⁺ T cells were purified from PBMCs by use of microbeads (Miltenyi Biotec). Naïve CD4⁺ T cells (10⁵/well) were cultured with regulatory T cells at a ratio of 10:1 in OKT3 (2 μg/ml)-coated, U bottomed 96-well plates containing the following ligands. LPS (100 ng/ml), imiquimod (10 μg/ml), loxoribine (500 μM), poly(I:C) (25 μg/ml), ssRNA40/LyoVec (3 μg/ml), ssRNA33/LyoVec (3 μg/ml), pam3CSK4 (200 ng/ml) and flagellin (10 μg/ml), all purchased from Invivogen (San Diego, CA). CpG-A (3 μg/ml), CpG-B (3 μg/ml) and poly-G oligonucleotides (3 μg/ml) were synthesized by Integrated DNA Technologies (Coralville, Iowa).

### Example 7

### Generation and Characterization of Breast Tumor-Infiltrating γδ T Cells

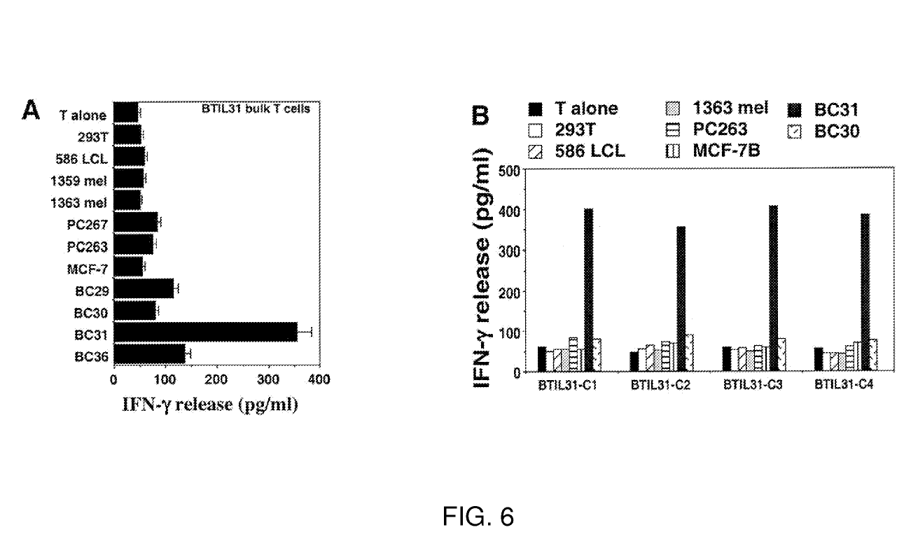

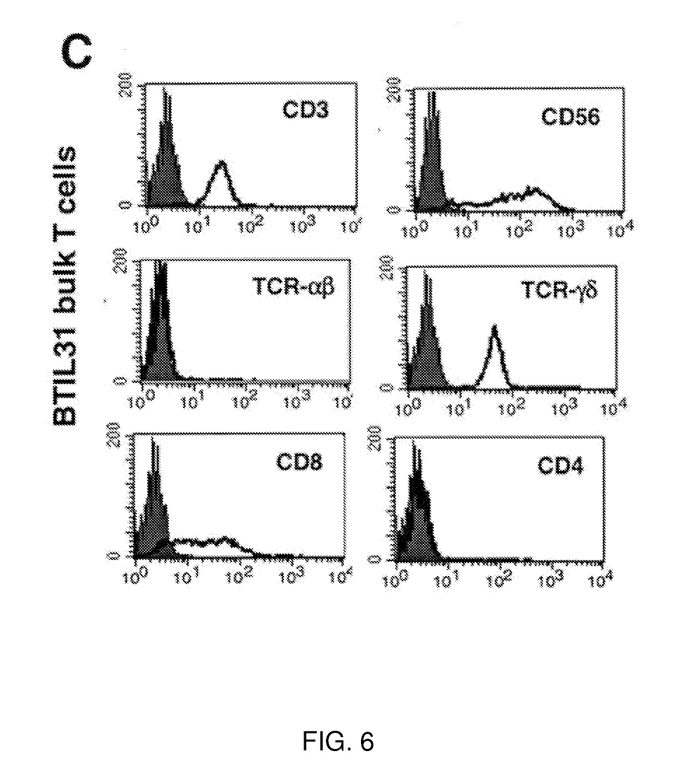

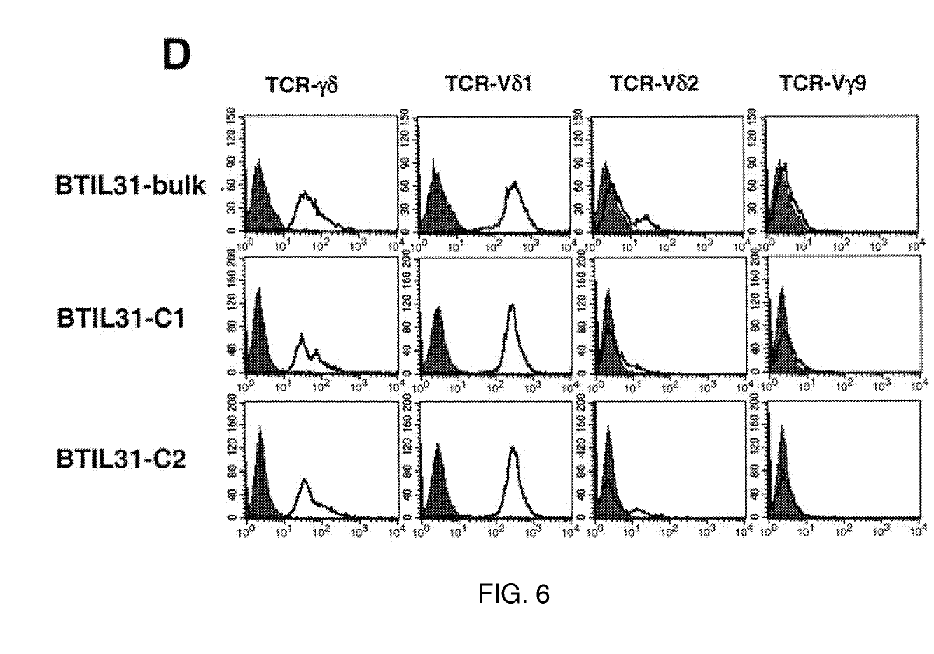

Forty-five fresh breast tumor samples were collected from which 25 breast tumor-infiltrating lymphocytes (TILs) were generated, some of which recognized autologous tumor cell lines. Results in FIG. 6A showed that BTIL31 specifically recognize autologous tumor cells (BC31), but did not respond to other allogeneic breast cancer cells (MCF-7, BC29, BC30 and BC36), prostate cells (PC263 and PC267), melanoma tumor cells (1363mel and 1359mel), 586 EBV-B cells or 293 T cells. Tumor reactivity and specificity were also observed with several T cell clones established from the bulk BTIL31 (FIG. 6B), indicating that BTIL31 and its clones are breast tumor-specific T cells.

To determine if T cell recognition by BTIL31 is restricted by MHC molecules, a T-cell functional assay was performed in the presence or absence of specific antibodies against HLA molecules. It was evident that none of these blocking antibodies could inhibit T cell recognition, indicating that unlike conventional CD4⁺ or CD8⁺ T cells, these BTIL31 T cells did not require MHC-class I or II molecules for tumor recognition. To determine the phenotype of BTIL31 cells, FACS analysis was performed, and these cells were positive for CD3, CD8, CD56 and TCR-γδ molecules, but negative for the αβ TCR marker (FIG. 6C), indicating that they were tumor-specific TCR-γδ T cells. It has been known that the TCR-γ9δ2 subset is a dominant population (Hayday and Tigelaar, 2003), representing 3-5% in the total peripheral T cells, while TCR-Vδ1 T cells are in epithelial tissues and skin. To determine the subtype of BTIL31 T cells, bulk BTIL31 line and its clones were stained with an anti-Vδ1 or anti-Vδ2 antibody, and found that more than 95% T cells were positive for Vδ1, but negative for Vδ2 antibody staining (FIG. 6D). Thus, it was concluded from these results that BTIL31 cells are breast tumor-specific γδ₁ T cells and predominantly accumulate in breast tumor tissues.

### Example 8

### Prevalence of Regulatory γ67 T Cell Population in Breast and Prostate Cancer

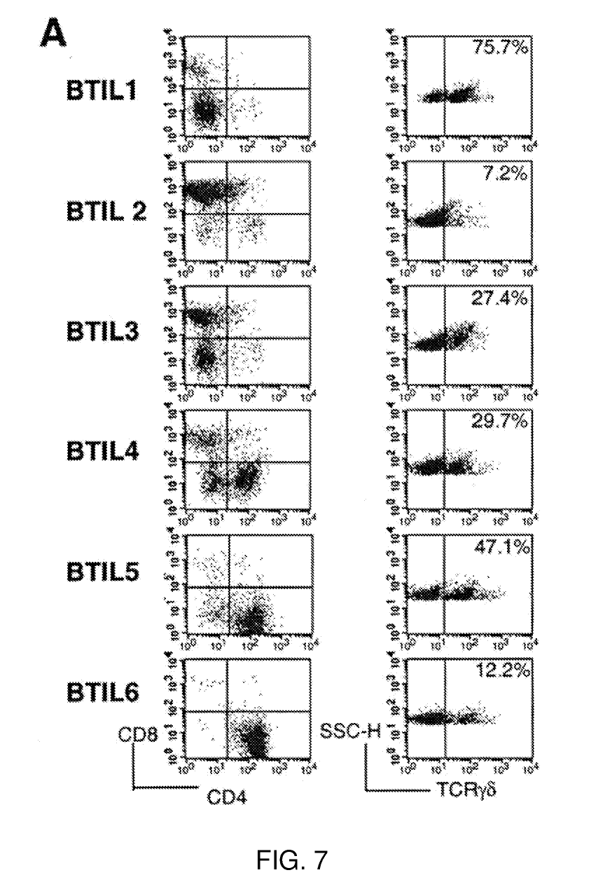

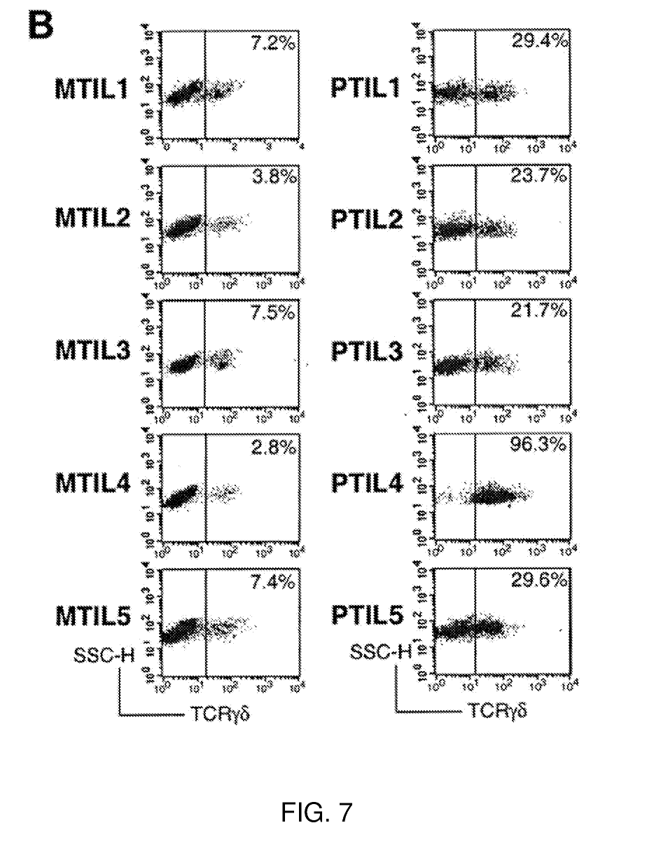

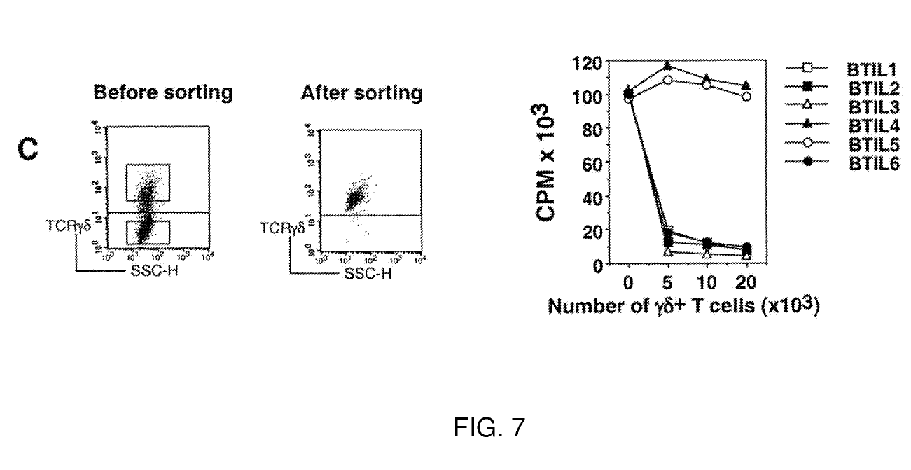

To determine if high percentages of γδ T cell population are also present in other breast cancer samples, additional ten breast tumor-derived TILs were analyzed, and most TILs contained a high percentage of γδ T cells. Representative data are shown in FIG. 7A. These results led to the findings in other tumors such as prostate cancer and melanoma. As shown in FIG. 7B, like breast tumor-derived TILs, prostate tumor-derived TILs also contained a high percentage of γδ T cells in the total T-cell population, whereas melanoma-derived TIL exhibited a low percentages of γδ T cells. It appears that an elevated proportion of γδ T cell population is prevalent in epithelium-originated tumors, including breast cancer.

### Example 9

### Tumor-Specific γδ 1 T Cells Suppress Naïve and Effector T Cell Function

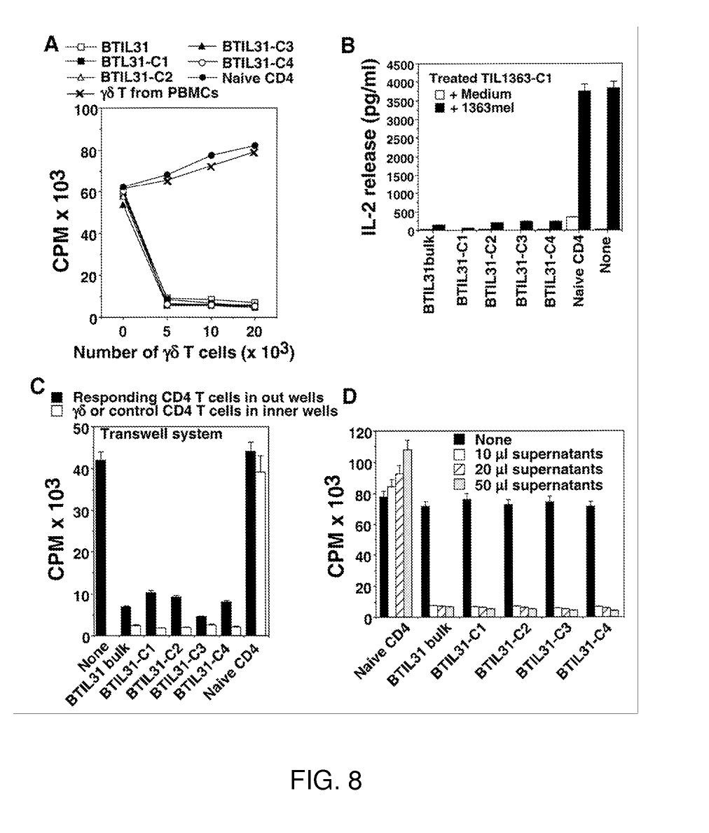

Although γδ1 T cells have been implicated in innate immunity as well as in antigen presentation, evidence for their role as regulatory T cells is still lacking. To test this possibility, a functional assay was performed similarly to those for CD4⁺ Treg cells and found that BTIL31 bulk cell line and its clones strongly inhibited the proliferation of naïve T cells (FIG. 8A). By contrast, naïve CD4⁺ T cells and γδ T cells isolated from fresh PBMCs enhanced, rather inhibited, the proliferation of naïve T cells in response to anti-CD3 stimulation. To test whether other breast tumor-derived γδ T cells possess the suppressive function, γδ⁺ T cells were purified from bulk TILs by FACS sorting after staining with anti-γδ antibody, and used to test for their ability to suppress naïve T cell proliferation (FIG. 7C). Like BTIL31 γδT cells, most breast tumor-derived γδ T cells possessed suppressive activity (FIG. 7C). These results strongly indicate that the majority of breast tumor-derived γδ⁺ cells possess a potent suppressive function shared by CD4⁺ Treg cells.

It was next tested whether these tumor specific BTIL31 γδ₁ T cells could inhibit IL-2 secretion from CD4⁺ or CD8⁺ effector T cells in response to TCR stimulation. As previously demonstrated (Wang and Wang, 2005), CD4⁺ T helper TIL1363 cells secreted large amounts of IL-2 after stimulation with 1363mel tumor cells (FIG. 8B). Co-culturing CD4⁺ T helper TIL1363 cells with OKT3-pretreated γδ1 T cells resulted in inhibition of IL-2 secretion from CD4⁺ TIL1363 T cells (FIG. 8B). By contrast, control CD4⁺ T cells and PBMC-derived γδ T cells failed to do so.

To determine the suppressive mechanism, transwell experiments were performed, and it was found that BTIL31 γδ1 Treg cells and its clones were capable of suppressing naïve T cell proliferation (FIG. 8C), suggesting that a cell-cell contact mechanism is not required for their suppression. Indeed, further experiments revealed that as little as 10 μl of BTIL31 cell supernatant was sufficient to inhibit more than 90% of the proliferative activity of naïve T cells, compared with that obtained without BTIL31 cell supernatant (FIG. 8D). Taken together, these results clearly indicate that BTIL31 γδ1 T cells have a potent regulatory function to suppress naïve T cell proliferation and IL-2 secretion of effector cells. They were therefore designated them BTIL31 γδ1 Treg cells hereafter.

### Example 10

### Cytokine Profiles and Phenotypic Marker Analysis of BTIL31 □□₁ T Cells

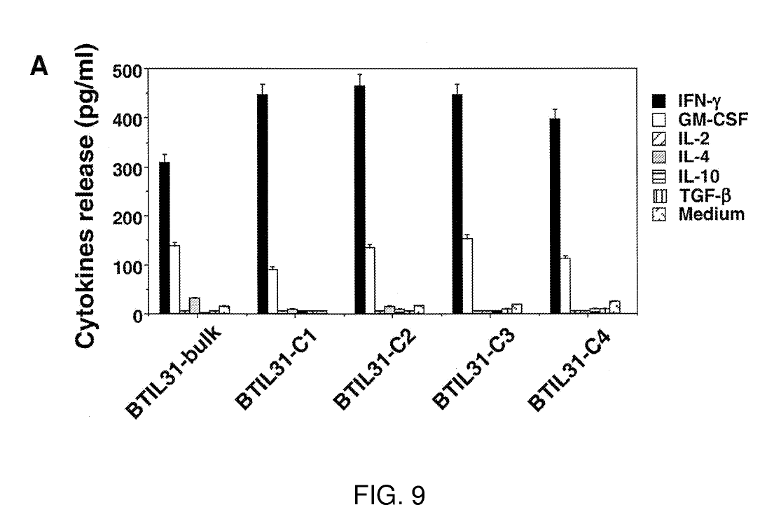

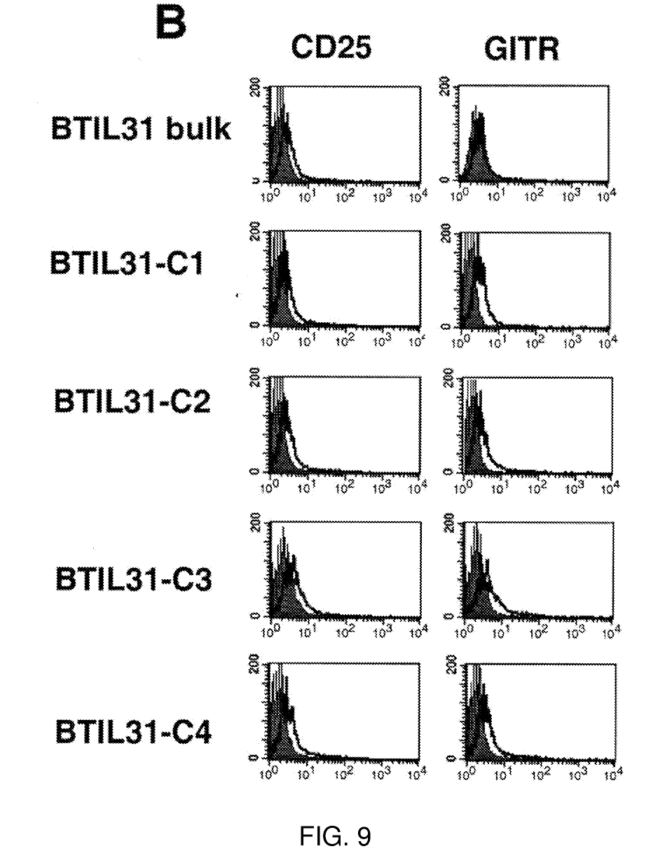

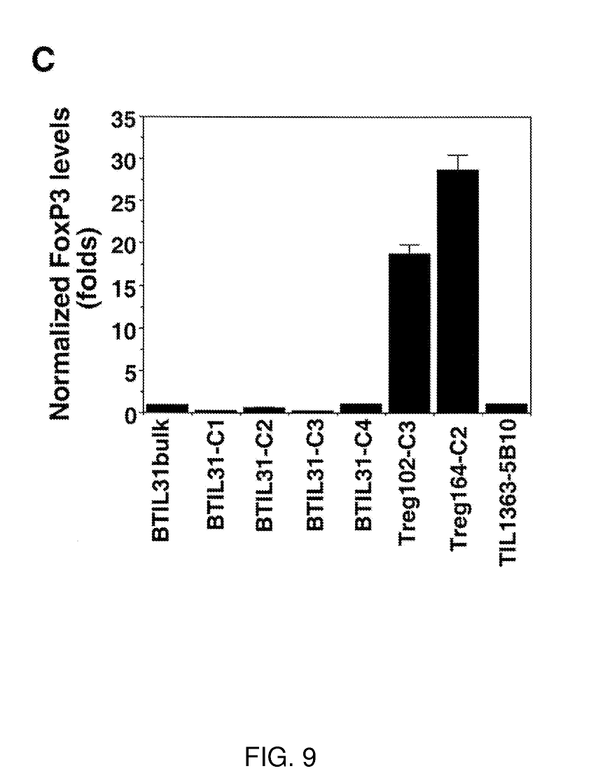

To determine whether BTIL31 γ67 1 Treg cells share any phenotypic properties with CD4⁺ Treg cells, the cytokine profiles and surface markers were examined. Cytokine profiling analysis showed that these γδ₁ T cells secreted IFN-γ and GM-CSF, but not other cytokines such as IL-2, IL-4, IL-10 or TGF-β when they were stimulated with either autologous tumor cells or an anti-CD3 antibody (FIG. 9A). To determine whether BTIL31 γδ1 T cells express surface markers typically found on CD4⁺ Treg cells, both FACS and real-time PCR analyses were performed for CD25, GITR and Foxp3. In sharp contrast to results from CD4⁺ Treg cells, BTIL31 γδ1 Treg cells were negative for CD25 and GITR (FIG. 9B). There was little or no Foxp3 expression in BTIL31 γδ1 Treg cells compared with that in previously characterized CD4⁺ Treg cell clones (FIG. 9C). These results indicate that BTIL31 γδ1 T cells secrete Th1-like cytokines and do not share the relatively specific markers of CD4⁺ Treg cells.

### Example 11

### Impairment of DC Maturation and Function by BTIL31 γδ1 Treg Cells

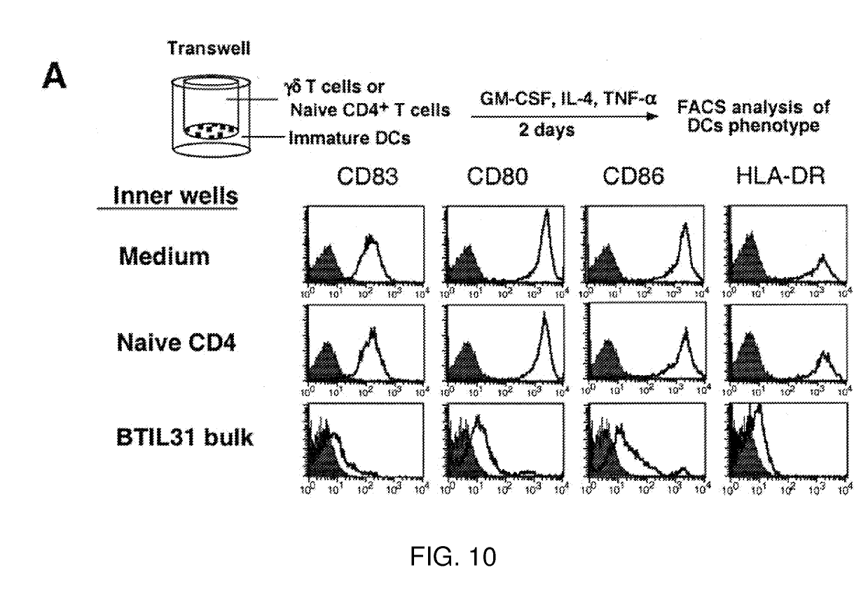

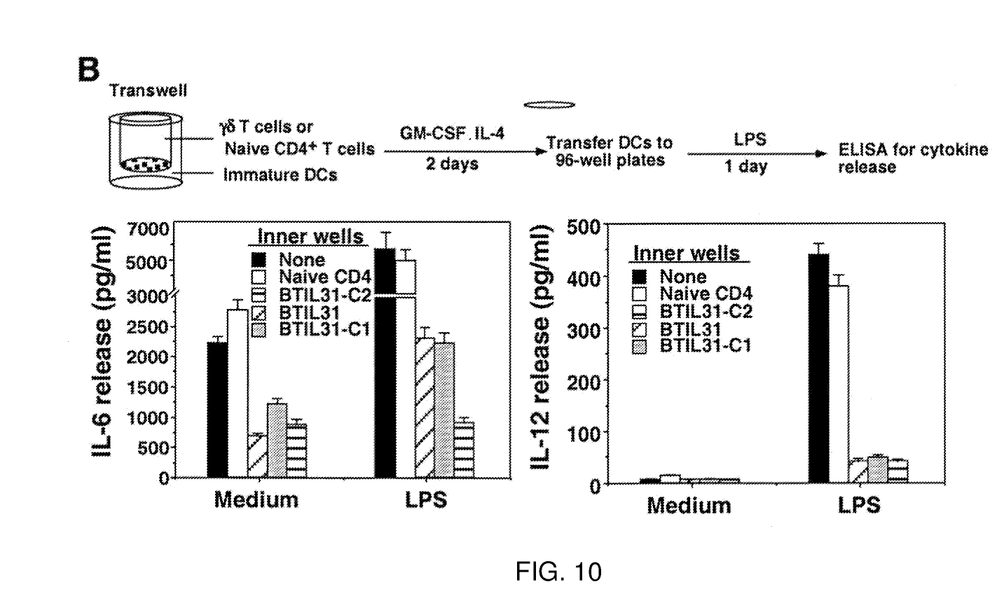

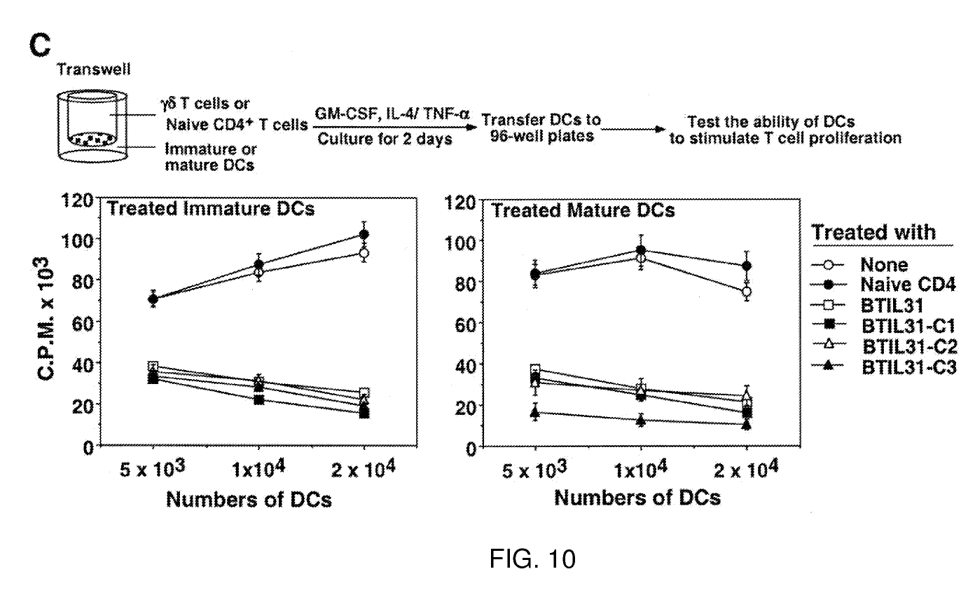

It was next tested whether BTIL31 γδ1 Treg cells could inhibit the maturation and function of DCs. Because BTIL31 γδ1 Treg cells mediated suppression through soluble factors, BTIL31 γδ1 Treg or control cells were cultured in the inner well and DCs in the outer wells of a transwell plate, and then the ability of DCs to mature in the presence of cytokines of IL-4, GM-CSF and TNFα was tested by FACS analysis based on the expression levels of CD83, CD80, CD86 and HLA-DR molecules. As shown in FIG. 10A, DCs treated with or without naïve CD4⁺ T cells expressed high levels of CD83, CD80, CD86 and HLA-DR molecules after being incubated with maturation cytokines. In sharp contrast, treatment with BTIL31 γδ1 Treg cells blocked DC maturation, in that expression levels of all maturation markers (CD83, CD80, CD86 and MHL-DR) were markedly inhibited. It wasnext tested whether BTIL31 γδ1 Treg cells could impair the function of DCs. After treatment with BTIL31 γδ1 Treg cells or naïve CD4⁺ T cells in a transwell plate for 18 h, the DCs were tested for their ability to respond to LPS. IL-6 and IL-12 release by DCs was determined by ELISA. As shown in FIG. 10B, the untreated mature DCs secreted large amounts of IL-6 and IL-12 in response to LPS. By contrast, the release of both cytokines from DCs treated with BTIL31 γδ1 Treg cells markedly decreased. Treatment with naïve CD4 T cells had no effect on cytokine release.

The ability of DCs to stimulate the proliferation of naïve T cells in response to soluble anti-CD3 antibody is well recognized (Shevach, 2002). To test whether BTIL31 γδ1 Treg cells can inhibit the ability of DCs to stimulate the proliferation of naïve T cells, naïve CD4⁺ T cells were co-cultured with different numbers of immature or mature DCs, which had been treated with BTIL31 γδ1 Treg cells. As shown in FIG. 10C, untreated DCs strongly stimulated the proliferation of naïve CD4⁺ T cells, regardless of their maturation statuses. However, neither immature nor mature DCs treated with BTIL31 γδ1 Treg cell culture supernatants could stimulate naïve T cell proliferation. The stimulating ability of DCs treated with naïve CD4⁺ T cells was not impaired. It was concluded from these results that BTIL31 γδ1 Treg cells not only can block the maturation of DCs, but can also suppress their ability to secrete cytokines in response to LPS.

### Example 12

### BTIL31 γδ1 Treg Cells Specifically Kill Tumor Cells Through a Trail-Mediated Mechanism

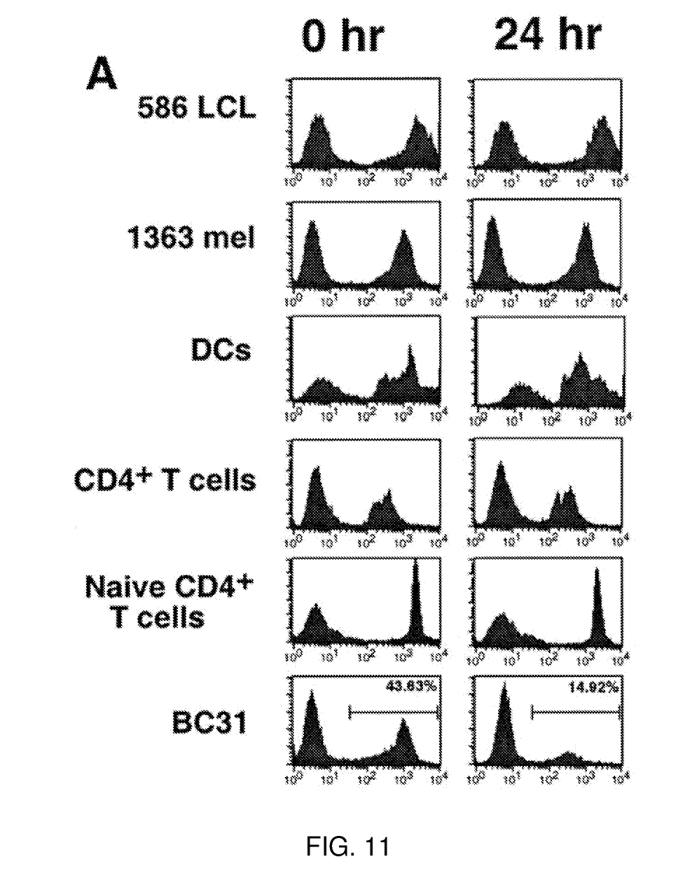

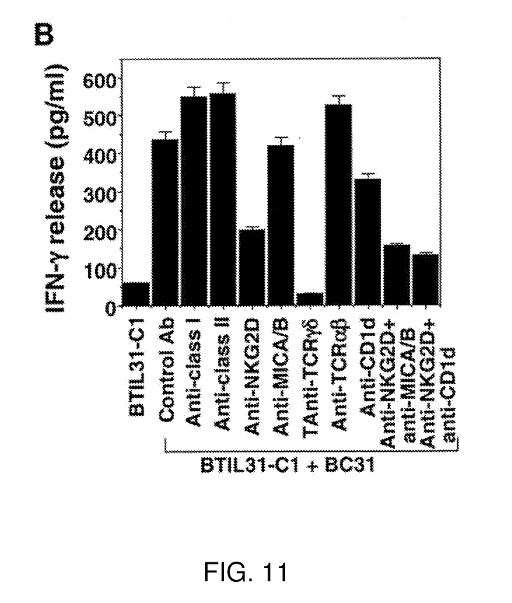

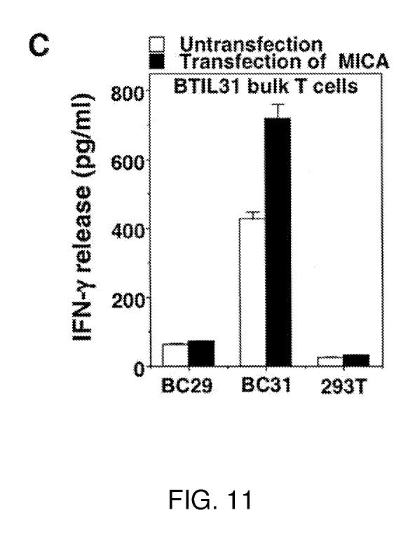

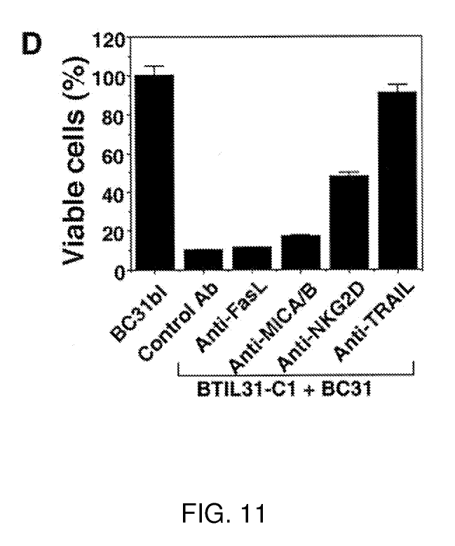

To exclude the possibility that the suppressive effects of BTIL31 γδ1 Treg cells on CD4⁺, CD8⁺ T cells as well as DCs are due to their cytotoxic activity on these target cells, naïve T cells were labeled with a low concentration of carboxyfluorescein diacetate succinimidyl ester (CFSE) as a reference and different target cells (586LCL, 1363mel, DCs, CD4⁺ T cells and naïve T cells) were labeled with a high concentration of CFSE. The mixture of CFSE-low T cells and CFSE-high target cells in a 1:1 ratio were then co-cultured with BTIL31 γδ1 Treg cells. After 24 h incubation, both CFSE-low and CFSE-high cell populations were analyzed by FACS. The relative numbers of both cell populations at 0 h served as a reference. As shown in FIG. 11A, the relative numbers of CFSE-high labeled 586LCL, 1363mel, DCs, CD4⁺ T cells and naïve T cells remained unchanged when compared with the corresponding cell numbers at 0 h, indicating that BTIL31 γδ1 Treg cells suppressed T cell proliferation as well as the maturation and stimulatory ability of DCs, but did not kill these cells. By contrast, the relative numbers of CFSE-high autologous BC31 tumor cells dramatically decreased after 24 h culture with BTIL31 γδ1 Treg cells, indicating that BTIL31 γδ1 Treg cells were capable of specifically recognizing and killing autologous tumor cells.

To determine how BTIL31 γδ1 Treg cells recognize BC31 tumor cells, it was attempted to block their ability to recognize BC31 tumor cells through various antibodies against MHC class I, class II, NKG2D, MICA/B, γδ or αβ TCR molecules. BTIL31 γδ1 Treg cells were cultured with the BC31 tumor cells in the absence or presence of various blocking antibodies and found that the recognition of tumor cells by BTIL31 γδ1 Treg cells was completed blocked by an anti-TCR γδ antibody, and partially blocked by an anti-NKG2D antibody alone or in combination with anti-MICA/B or anti-CD1d antibody (FIG. 11B). By contrast, little or no inhibition was observed with anti-MICA/B, anti-CD1d, anti-MHC class I, anti-MHC class II or control antibodies (FIG. 11B). These results indicate that tumor recognition by BTIL31 γδ1 Treg cells require the interaction between TCR and an unknown antigen, while NKG2D may enhance T cell recognition by interacting with MICA. To test this possibility, HEK293T, BC29 and BC31 cells were transfected with a MICA cDNA and then evaluated T cell recognition of these target cells on the basis of IFN-γ release from BTIL31 γδ1 Treg cells. T cell recognition was significantly enhanced by BC31 cells transfected with MICA compared with BC31 cells without MICA. However, MICA expression alone in BC29 and 293T cells failed to activate BTIL31 γδ1 Treg cells (FIG. 11C). These results are consistent with data in FIG. 11B, further supporting the notion that the interaction between of TCR and antigens expressed on tumor cells is necessary and sufficient for T cell activation.

It was next determined how BTIL31 γδ1 Treg cells kill BC31 tumor cells. Since T cells can kill target cells through either a TNF family molecules such as TNF, FAS or TRAIL (TNF-related apoptosis inducing ligand) mediated apoptosis, or perforin/granzyme-mediated killing mechanisms (Hayday and Tigelaar, 2003; Pennington et al., 2005), BTIL31 γδ1 Treg cells were co-cultured with BC31 tumor cells in the absence or presence of anti-FasL, anti-TRAIL, anti-MICA/B and anti-NKG2D antibodies, and counted visible cells after 12 h incubation. As shown in FIG. 11D, anti-TRAIL antibody almost completely blocked the killing of targets, while anti-NKG2D antibody partially inhibited the tumor cell killing by BTIL31 γδ1 Treg cells. By contrast, antibodies against FasL and MICA/B had little or no effect on blocking tumor killing activity. These results demonstrated that BTIL31 γδ1 Treg cells specifically killed BC31 tumor cells through a TRAIL-dependent mechanism.

### Example 13

### T Cell Activation is Required for Up-Regulation and Production of Trail

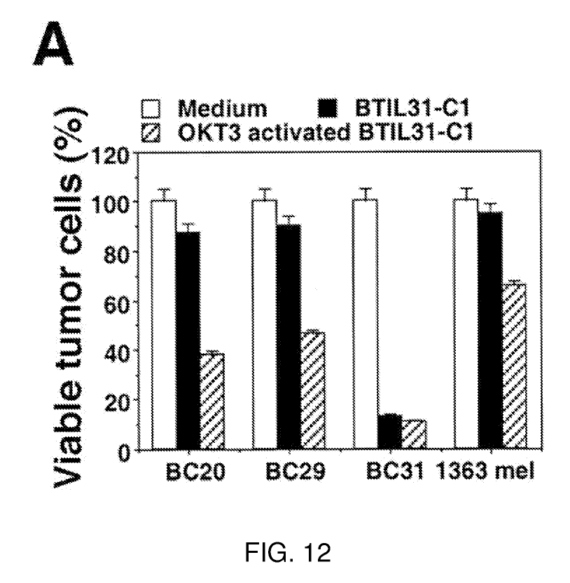

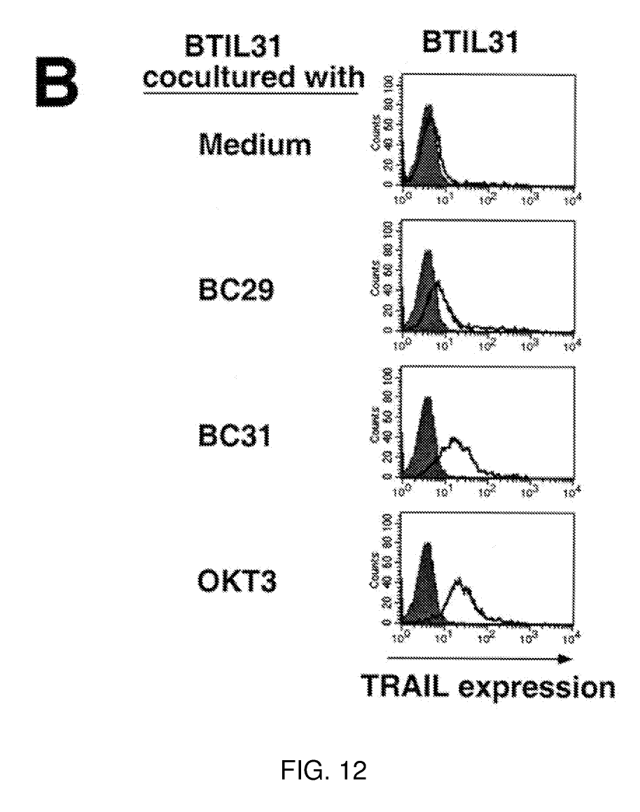

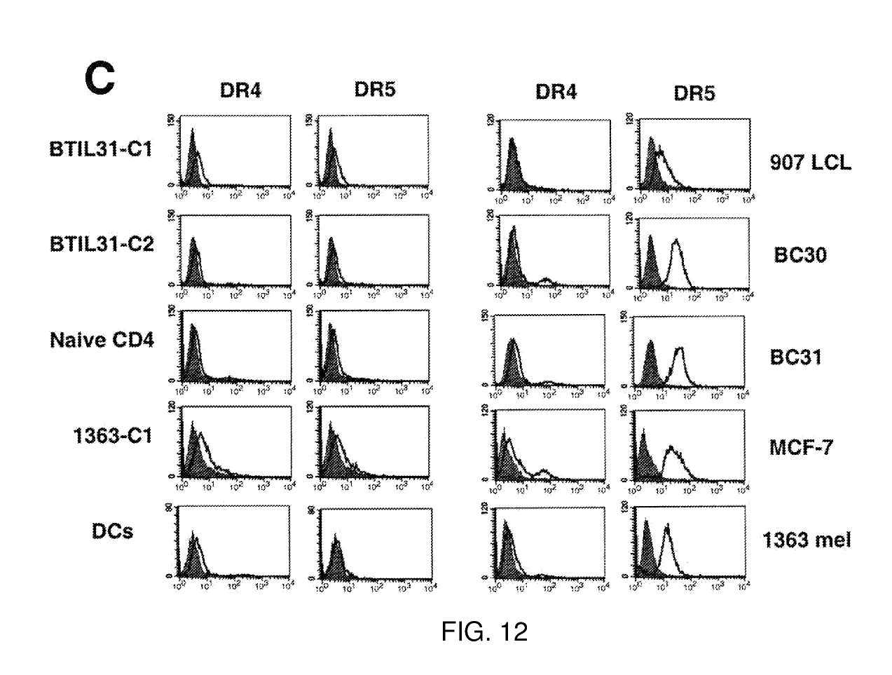

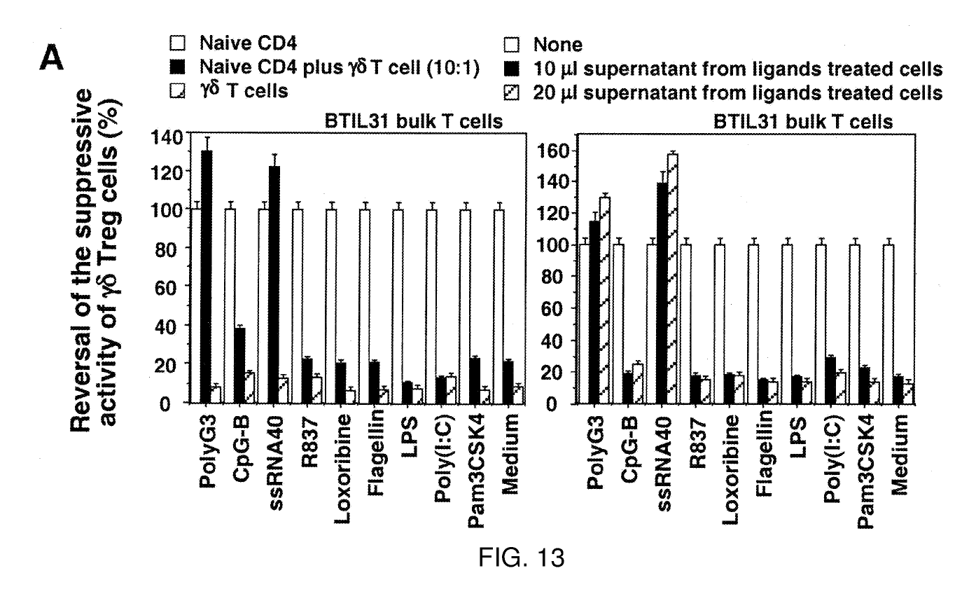

To understand why BTIL31 γδ1 Treg cells can only kill the autologous BC31 cells, but not other tumor cell lines, it was reasoned that T cell activation is prerequisite for the up-regulation and production of TRAIL. To test this possibility, BTIL31 γδ1 Treg cells were pre-activated with anti-CD3 (OKT3) antibody. After washing with PBS, OKT3-activated or untreated BTIL31 γδ1 Treg cells were co-cultured with various tumor cells to test their ability to kill target cells. As shown in FIG. 12A, OKT3-activated, but not untreated, BTIL31 γδ1 Treg cells could kill all tumor cell lines, while untreated BTIL31 γδ1 Treg cells only killed autologous BC31 cells as expected. To determine the expression level of TRAIL following OKT3 stimulation, FACS analysis was performed for BTIL31 γδ1 Treg cells. BTIL31 γδ1 Treg cells did not express TRAIL molecules without OKT3 stimulation or weakly expressed TRAIL after culturing with BC29 cells. By contrast, TRAIL expression was significantly increased after stimulation either with BC31 tumor cells or an OKT3 antibody (FIG. 12B), suggesting that T cell activation with autologous tumor cells or OKT3 is prerequisite for expression and production of TRAIL, which, in turn, mediated apoptosis of tumor cells.

To determine why activated BTIL31 γδ1 Treg cells induce apoptosis of tumor cells, but not T cells and DCs, the expression of TRAIL receptors—DR4 and DR5 molecules was examined, and it was found that all breast tumor cell lines (BC30, BC29, MCF-7) and melanoma 1363mel cells expressed high levels of DR5, but not DR4 molecules (FIG. 12C). EBV-transformed 907LCL cells expressed a low level of DR5, but not DR4. Neither DR4 nor DR5 molecules were expressed in T cells and DCs (FIG. 12C). Thus, DR5 expression on tumor cells may account for TRAIL-mediated apoptosis of tumor cells, but not T cells or DCs, by activated BTIL31 γδ1 Treg cells.

### Example 14

### TLR-8 Signaling Controls γδ1 Treg Cell Suppressive Function, but not Trail Activity

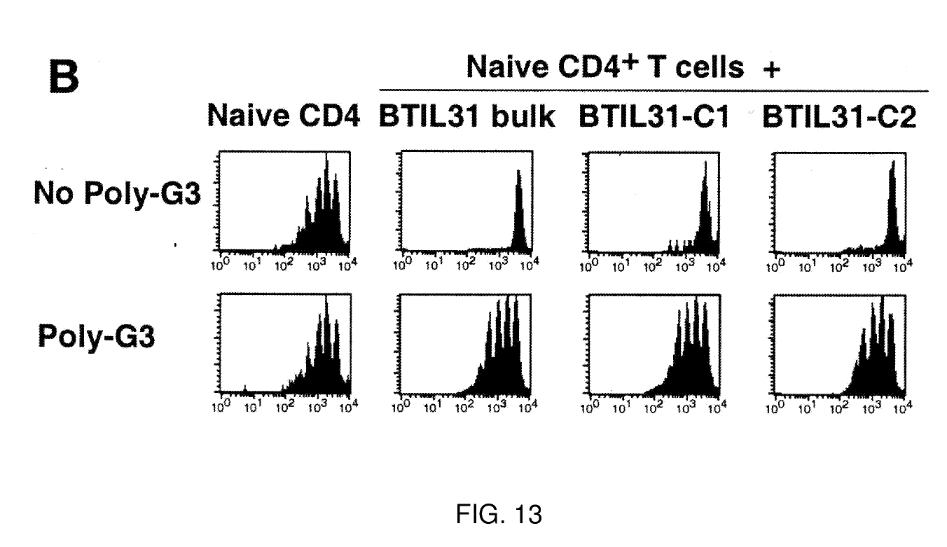

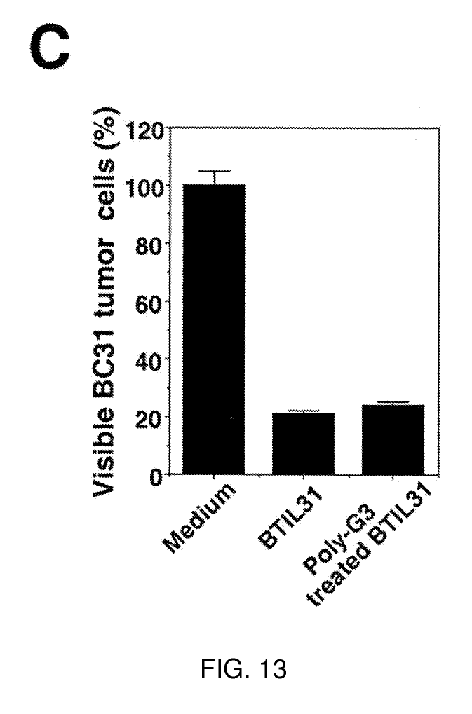

It was recently demonstrated that poly-guanosine (poly-G) oligonucleotides could directly reverse the suppressive function of both antigen-specific Treg102 cells as well as naturally occurring CD4⁺CD25⁺ Treg cells (Peng et al., 2005). To test whether the suppressive function of BTIL31 γδ1 Treg cells could be reversed by TLR-8 ligands, cells were treated with a panel of TLR ligands and then test their ability to suppress naïve T cell proliferation. As shown in FIG. 13A, TLR-8 ligands (poly-G3 and ssRNA40), but not ligands for other TLRs, could reverse the suppressive function of BTIL31 γδ1 Treg cells and restored the proliferation of naive CD4⁺ T cells. Since BTIL31 γδ1 Treg cells mediated immune suppression through soluble factors in cell supernatants, it was tested whether the supernatants harvested from the Poly-G3 or ssRNA40 treated γδ1 Treg cells became nonsuppressive. Indeed, the supernatants harvested from the Poly-G3 or ssRNA40 treated γδ1 Treg cells enhanced rather than inhibited the proliferation of naïve CD4⁺ T cells, in sharp contrast to the supernatants from untreated or treated with other TLR ligands, which retained potent suppressive activity (FIG. 13A). To further determine whether poly-G3 or ssRNA40 treatment restored the proliferation or division of naïve T cells, but not γδ1 Treg cells, BTIL31 γδ1 Treg cells were cultured with CFSE-labeled naïve CD4⁺ T cells in the presence or absence of Poly-G3. After 48 h, CFSE-labeled cells were gated for FACS analysis. As shown in FIG. 13B, both BTIL31 bulk Treg cells and clones strongly inhibited the division of naïve CD4⁺ T cells compared with naïve T cells without Treg cells. However, treatment of poly-G3 oligonucleotides completely restored the proliferation or division of naive T cells. These results indicate that TLR-8 signaling pathway is a common regulatory mechanism shared by γδ1 Treg cells and CD4⁺ Treg cells for controlling their suppressive function.

To test whether TLR-8 signaling pathway is involved in the control of TRAIL-mediated killing of tumor cells, BTIL31 γδ1 Treg cells were treated with or without poly-G3 oligonucleotides and were tested for their ability to kill BC31 tumor cells. As shown in FIG. 13C, treatment of BTIL31 γδ1 Treg cells with poly-G3 oligonucleotides has no effect on their killing ability of tumor cells, suggesting that TRAIL expression and its killing ability of tumor cells is not linked to the reversal of Treg cell suppressive function.

### Example 15

### Significance of Tumor Infiltrating γδ Treg Cells and Their Functional Regulation

Elevated percentage of CD4⁺ CD25⁺ Treg cells have been detected either at tumor sites or peripheral blood of patients with different types of cancers, and thus were generally thought to play a major role in suppressing immune responses. Although an earlier study showed the presence of high percentage of CD4⁺ CD25⁺ Treg cells in breast cancer (Liyanage et al., 2002), no significant high percentages of CD4⁺ CD25⁺ Treg cells were identified in breast tumor-derived TILs, which was consistent with a recent report showing no difference in the percentages of CD4⁺ CD25⁺ Treg cells between cancer patients and healthy donors (Okita et al., 2005). These results imply that other subsets of Treg cells may play a critical role in suppressing immune responses at tumor sites. Indeed, it was demonstrated that both prostate and breast tumor-derived TILs contained a dominant γδ T cell population. Among subsets of γδ T cell population, it was found that tumor-derived γδ T cells were exclusively γδ1 subset, but little or no γ9δ2 T cells, a dominant population normally found in the peripheral blood. Although γ9δ2 T cells have been demonstrated to function as innate immune cells against bacteria and viruses as well as antigen-presenting cells (APCs), the studies showed that most breast tumor-derived γδ₁ T cells functioned as Treg cells to suppress the proliferation and IL-2 secretion of naïve/effector T cells and inhibit DC maturation and function. Of four breast tumor-derived γδ₁ Treg cell lines tested, all of them suppressed immune responses through previously unknown soluble factors other than IL-10 or TGF-β, indicating that a cell-contact is not required for their suppressive function. Taken together, these results collectively indicate that breast tumor-derived γδ₁ Treg cells shared some suppressive function as CD4⁺ CD25⁺ Treg cells, but they have their unique or distinct phenotypic and functional features. For example, tumor-derived γδ₁ Treg cells do not express CD25 and Foxp3 markers, typically expressed on CD4⁺ Treg cells. The dominant suppressive mechanism of CD4⁺ CD25⁺ Treg cells requires a cell-cell contact-dependent mechanism, while most tumor-derived γδ₁ Treg cells suppress immune responses through a soluble factor-dependent mechanism independent of IL-10 and/or TGF-β.

These findings raise an intriguing question regarding the mechanism by which γδ₁ Treg cells are recruited and accumulated in breast tumors. First, recent studies have linked chronic inflammation to cancer development and progression (Coussens and Werb, 2002; Greten et al., 2004; Karin and Greten, 2005). It has been suggested that proinflammatory cytokines (IL-6) and chemokines such as CCL22 secreted by tumor cells and immune cells recruit CD4⁺ Treg cells to tumor sites, but also favor the conversion of nonsuppressive T cells into Treg cells with suppressive function (Curiel et al., 2004; Huang et al., 2006). Whether γδ₁ Treg cells use a similar mechanism to infiltrate into tumor sites remains unknown. In an alternative embodiment, tumor cells may actively activate and expand γδ₁ Treg cells by either directly or indirectly presenting antigenic peptides for their recognition. In previous studies, tumor cells expressed tumor-specific antigens such as LAGE1 and ARTC1 and directly stimulate antigen-specific Treg cells (Wang et al., 2004; Wang et al., 2005). Since these breast tumor-derived γδ₁ Treg cells are capable of recognizing autologous tumor cells, it is reasonable to believe that tumor antigens expressed by breast tumor cells may play a critical role in the activation and maintenance of γδ₁ Treg cells at tumor sites.

However, activation of γδ₁ Treg cells by breast tumor cells led to the up-regulation and production of TRAIL, which mediated tumor cell apoptosis. In fact, it was found that most conventional (CD4⁺ and CD8⁺ αβ T cells) and unconventional γδ T cells can be up-regulated to produce TRAIL upon their activation by tumor cells or an anti-CD3 (OKT3) antibody, indicating that the secretion of TRAIL molecule is secondary to T cell activation through antigen-specific recognition of tumor cells. Thus, in certain embodiments of the invention tumor-specific γδ₁ T cells are an innate immune component of immunosurveillance for initial tumor destruction and growth control through the production soluble TRAIL after exposure to tumor cells. However, there is a continuous dynamic battle between immune cells (including γδ T cells) and tumor cells, and tumor cells eventually grow up even in the presence of TRAIL-mediated tumor apoptosis. The failure of such initial immune responses to control tumor cell growth leads to chronic inflammation and production of suppressive and proinflammatory cytokines such as TGF-β, IL-10, IL-6, and TNFα by tumor cells and tumor-infiltrating immune cells. The suppressive tumor microenvironment may favor the conversion of tumor-specific γδ₁ effector T cells to γδ Treg or suppressive cells, while keeping their antigen specificity and the ability to secrete TRAIL molecules unchanged. One similar example is tumor-associated macrophages (TAMs), which display a dual function: they can kill tumor cells following activation by IL-2 and IL-12, but can also promote tumor growth (Coussens and Werb, 2002; Condeelis and Pollard, 2006). Thus, γδ T cells can either inhibit or promote tumor growth, depending upon the tumor microenvironment.

Regardless of how γδ₁ Treg cells are generated near the breast tumor sites, either the depletion of γδ₁ Treg cells or the reversal of their function will be critical to enhance antitumor immune responses. Since γδ₁ Treg cells do not express a high level of CD25 molecule, anti-CD25 antibody or IL-2/toxin fusion protein (Ontak) cannot be used for this purpose. It was recently demonstrated that TLR8 signaling could reverse the suppressive function of naturally occurring CD4⁺ Treg as well as tumor-specific Treg cells (Peng et al., 2005). TLR8 ligands could also reverse the suppressive function of γδ₁ Treg cells, implying that γδ₁ Treg cells share a common suppressive mechanism, which can be reversed by triggering TLR8 signaling pathway. However, TLR8 treatment did not change the ability of γδ₁ T cells to secrete TRAIL molecule upon their activation through tumor-specific recognition or OKT3 stimulation, indicating that TLR8 signaling is linked to the function of soluble factors, but not TRAIL molecule.

Certain aspects of the present invention may be further characterized by suitable means in the art, of which the skilled artisan is aware. For example, it is further characterized how TLR8 signaling pathway controls the function of γδ₁ T cell secreted soluble suppressive factors and why the ligands for other TLRs failed to modulate the suppressive function of γδ₁ Treg cells. One plausible explanation is that expression pattern of TLRs on Treg cells may partially account for the specificity of TLR8-mediated functional reversal of Treg cells since human Treg cells consistently express high level of TLR8, but little or no TLR7 and TLR9 42. TLR8 expression has been detected in γδ₁ T cells. An alternative is that the downstream pathway of TLR8-MyD88 may differ from other TLRs. This notion was supported by a recent study, showing that stimulation of human CD4⁺ Treg cells with TLR5 ligands did not reverse their suppressive function, but rather enhance their suppressive function and Foxp3 expression (Crellin et al., 2005). It should be noted that since TLR8 is not functional in mice (Jurk et al., 2002), the regulation of murine Treg cells through TLR signaling may differ from the functional control of human Treg cells. Indeed, it was recently reported that TLR2 promoted the proliferation of both murine CD4⁺ Treg and effector cells, and transiently abrogated the suppressive function of murine CD4⁺ Treg cells (Sutmuller et al., 2006; Liu et al., 2006). Although murine γδ T cells have been implicated in the induction of tumor tolerance, these cells have not been isolated and characterized so far. Thus, it is not known whether the TLR2 ligands affect the function of murine γδ Treg cells. Since TLR8 ligands can modulate the function of all Treg cells, including CD4⁺, CD8⁺ and γδ₁ Treg cells, in specific aspects of the invention manipulation of Treg cell suppressive function, effector T cells and DCs by various TLR ligands allows one to tip the balance toward antitumor immunity. This new strategy is useful at least in improving the efficacy of vaccines directed to cancers and perhaps infectious diseases.

### REFERENCES

All patents, patent applications, and publications mentioned in the specifications are indicative of the levels of those skilled in the art to which the invention pertains. All patents, patent applications, and publications are herein incorporated by reference to the same extent as if each individual publication was specifically and individually indicated to be incorporated by reference.

### PATENTS AND PATENT APPLICATIONS

U.S. Provisional Application Ser. No. 60/660,028

PCT Patent Application PCT/US06/08379

U.S. Pat. No. 6,977,245

U.S. Pat. No. 6,239,116

U.S. Pat. No. 5,466,468

U.S. Pat. No. 6,400,487

### PUBLICATIONS

Arlen, P. M., Gulley, J. L., Parker, C., Skarupa, L., Pazdur, M., Panicali, D., Beetham, P., Tsang, K. Y., Grosenbach, D. W., Feldman, J., et al. 2006. A randomized phase II study of concurrent docetaxel plus vaccine versus vaccine alone in metastatic androgen-independent prostate cancer. Clin Cancer Res 12:1260-1269.

Attia, P., Maker, A. V., Haworth, L. R., Rogers-Freezer, L., and Rosenberg, S. A. 2005. Inability of a fusion protein of IL-2 and diphtheria toxin (Denileukin Diftitox, DAB389IL-2, ONTAK) to eliminate regulatory T lymphocytes in patients with melanoma. J Immunother 28:582-592.

Baecher-Allan, C., and Anderson, D. E. 2006. Immune regulation in tumor-bearing hosts. Curr Opin Immunol.

Bauer, S. et al. Activation of NK cells and T cells by NKG2D, a receptor for stress-inducible MICA. Science 285, 727-9 (1999).

Bienvenu, B., Martin, B., Auffray, C., Cordier, C., Becourt, C., and Lucas, B. 2005. Peripheral CD8⁺CD25⁺ T lymphocytes from MHC class II-deficient mice exhibit regulatory activity. J Immunol 175:246-253.

Boismenu, R. & Havran, W. L. Modulation of epithelial cell growth by intraepithelial gamma delta T cells. Science 266, 1253-5 (1994).

Brandes, M., Willimann, K. & Moser, B. Professional antigen-presentation function by human gammadelta T Cells. Science 309, 264-8 (2005).

Brenner, M. B. et al. Identification of a putative second T-cell receptor. Nature 322, 145-9 (1986).

Bukowski, J. F., Morita, C. T. & Brenner, M. B. Human gamma delta T cells recognize alkylamines derived from microbes, edible plants, and tea: implications for innate immunity. Immunity 11, 57-65 (1999).

Caron, G., Duluc, D., Fremaux, I., Jeannin, P., David, C., Gascan, H., and Delneste, Y. 2005. Direct stimulation of human T cells via TLR5 and TLR7/8: flagellin and R-848 up-regulate proliferation and IFN-gamma production by memory CD4⁺ T cells. J Immunol 175:1551-1557.

Cerwenka, A. et al. Retinoic acid early inducible genes define a ligand family for the activating NKG2D receptor in mice. Immunity 12, 721-7 (2000).

Condeelis, J. & Pollard, J. W. Macrophages: obligate partners for tumor cell migration, invasion, and metastasis. Cell 124, 263-6 (2006).

Constant, P. et al. Stimulation of human gamma delta T cells by nonpeptidic mycobacterial ligands. Science 264, 267-70 (1994).

Cosmi, L., Liotta, F., Lazzeri, E., Francalanci, M., Angeli, R., Mazzinghi, B., Santarlasci, V., Manetti, R., Vanini, V., Romagnani, P., et al. 2003. Human CD8⁺CD25⁺ thymocytes share phenotypic and functional features with CD4⁺CD25⁺ regulatory thymocytes. Blood 102:4107-4114.

Coussens, L. M., and Werb, Z. 2002. Inflammation and cancer. Nature 420:860-867.

Crellin, N. K., Garcia, R. V., Hadisfar, O., Allan, S. E., Steiner, T. S., and Levings, M. K. 2005. Human CD4⁺ T cells express TLR5 and its ligand flagellin enhances the suppressive capacity and expression of FOXP3 in CD4⁺CD25⁺ T regulatory cells. J Immunol 175:8051-8059 (2005).

Curiel, T. J., Coukos, G., Zou, L., Alvarez, X., Cheng, P., Mottram, P., Evdemon-Hogan, M., Conejo-Garcia, J. R., Zhang, L., Burow, M., et al. 2004. Specific recruitment of regulatory T cells in ovarian carcinoma fosters immune privilege and predicts reduced survival. Nat Med 10:942-949.

Dannull, J., Su, Z., Rizzieri, D., Yang, B. K., Coleman, D., Yancey, D., Zhang, A., Dahm, P., Chao, N., Gilboa, E., et al. 2005. Enhancement of vaccine-mediated antitumor immunity in cancer patients after depletion of regulatory T cells. J Clin Invest 115:3623-3633.

Diefenbach, A., Jamieson, A. M., Liu, S. D., Shastri, N. & Raulet, D. H. Ligands for the murine NKG2D receptor: expression by tumor cells and activation of NK cells and macrophages. Nat Immunol 1, 119-26. (2000).

Diefenbach, A., Jensen, E. R., Jamieson, A. M. & Raulet, D. H. Rael and H60 ligands of the NKG2D receptor stimulate tumour immunity. Nature 413, 165-71. (2001).

Dudley, M. E., Wunderlich, J. R., Robbins, P. F., Yang, J. C., Hwu, P., Schwartzentruber, D. J., Topalian, S. L., Sherry, R., Restifo, N. P., Hubicki, A. M., et al. 2002. Cancer regression and autoimmunity in patients after clonal repopulation with antitumor lymphocytes. Science 298:850-854.

Dunn, G. P., Old, L. J. & Schreiber, R. D. The three Es of cancer immunoediting. Annu Rev Immunol 22, 329-60 (2004).

Fisch, P. et al. Recognition by human V gamma 9/V delta 2 T cells of a GroEL homolog on Daudi Burkitt's lymphoma cells. Science 250, 1269-73 (1990).

Fontenot, J. D., Gavin, M. A., and Rudensky, A. Y. 2003. Foxp3 programs the development and function of CD4(⁺)CD25(⁺) regulatory T cells. Nat Immunol 4:330-336.

Fontenot, J. D., Rasmussen, J. P., Williams, L. M., Dooley, J. L., Farr, A. G., and Rudensky, A. Y. 2005. Regulatory T cell lineage specification by the forkhead transcription factor foxp3. Immunity 22:329-341.

Gavin, M. A., Torgerson, T. R., Houston, E., Deroos, P., Ho, W. Y., Stray-Pedersen, A., Ocheltree, E. L., Greenberg, P. D., Ochs, H. D., and Rudensky, A. Y. 2006. Single-cell analysis of normal and FOXP3-mutant human T cells: FOXP3 expression without regulatory T cell development. Proc Natl Acad Sci USA 103:6659-6664.

Girardi, M. et al. Regulation of cutaneous malignancy by gammadelta T cells. Science 294, 605-9 (2001).

Girardi, M. et al. Resident skin-specific gammadelta T cells provide local, nonredundant regulation of cutaneous inflammation. J Exp Med 195, 855-67 (2002).

Gober, H. J. et al. Human T cell receptor gammadelta cells recognize endogenous mevalonate metabolites in tumor cells. J Exp Med 197, 163-8 (2003).

Greten, F. R. et al. IKKbeta links inflammation and tumorigenesis in a mouse model of colitis-associated cancer. Cell 118, 285-96 (2004).

Greten, F. R., Eckmann, L., Greten, T. F., Park, J. M., Li, Z. W., Egan, L. J., Kagnoff, M. F., and Karin, M. 2004. IKKbeta links inflammation and tumorigenesis in a mouse model of colitis-associated cancer. Cell 118:285-296.

Groh, V. et al. Broad tumor-associated expression and recognition by tumor-derived gamma delta T cells of MICA and MICB. Proc Natl Acad Sci USA 96, 6879-84 (1999).

Groh, V., Steinle, A., Bauer, S. & Spies, T. Recognition of stress-induced MHC molecules by intestinal epithelial gammadelta T cells. Science 279, 1737-40 (1998).

Havran, W. L. A role for epithelial gammadelta T cells in tissue repair. Immunol Res 21, 63-9 (2000).

Hayday, A. & Tigelaar, R. Immunoregulation in the tissues by gammadelta T cells. Nat Rev Immunol 3, 233-42 (2003).

Hayday, A. C. [gamma][delta] cells: a right time and a right place for a conserved third way of protection. Annu Rev Immunol 18, 975-1026 (2000).

Hori, S., Nomura, T., and Sakaguchi, S. 2003. Control of regulatory T cell development by the transcription factor foxp3. Science 299:1057-1061.

Houghton, A. N., Gold, J. S., and Blachere, N. E. 2001. Immunity against cancer: lessons learned from melanoma. Curr Opin Immunol 13:134-140.

Hu, D., Ikizawa, K., Lu, L., Sanchirico, M. E., Shinohara, M. L., and Cantor, H. 2004. Analysis of regulatory CD8 T cells in Qa-1-deficient mice. Nat Immunol 5:516-523.

Huang, B., Pan, P. Y., Li, Q., Sato, A. I., Levy, D. E., Bromberg, J., Divino, C. M., and Chen, S. H. 2006. Gr-1⁺CD115⁺ immature myeloid suppressor cells mediate the development of tumor-induced T regulatory cells and T-cell anergy in tumor-bearing host. Cancer Res 66:1123-1131 (2006).

Jameson, J. et al. A role for skin gammadelta T cells in wound repair. Science 296, 747-9 (2002).

Jameson, J., Witherden, D. & Havran, W. L. T-cell effector mechanisms: gammadelta and CD1d-restricted subsets. Curr Opin Immunol 15, 349-53 (2003).

Jarvis, L. B., Matyszak, M. K., Duggleby, R. C., Goodall, J.C., Hall, F. C., and Gaston, J. S. 2005. Autoreactive human peripheral blood CD8⁺ T cells with a regulatory phenotype and function. Eur J Immunol 35:2896-2908.

Jiang, H., and Chess, L. 2000. The specific regulation of immune responses by CD8⁺ T cells restricted by the MHC class 1b molecule, Qa-1. Annu Rev Immunol 18:185-216.

Jiang, H., and Chess, L. 2004. An integrated view of suppressor T cell subsets in immunoregulation. J Clin Invest 114:1198-1208.

Jones, E., Dahm-Vicker, M., Simon, A. K., Green, A., Powrie, F., Cerundolo, V., and Gallimore, A. 2002. Depletion of CD25⁺ regulatory cells results in suppression of melanoma growth and induction of autoreactivity in mice. Cancer Immun 2:1.

Jurk, M., Heil, F., Vollmer, J., Schetter, C., Krieg, A.M., Wagner, H., Lipford, G., and Bauer, S. 2002. Human TLR7 or TLR8 independently confer responsiveness to the antiviral compound R-848. Nat Immunol 3:499 (2002).

Kabelitz, D., Marischen, L., Oberg, H. H., Holtmeier, W. & Wesch, D. Epithelial defence by gamma delta T cells. Int Arch Allergy Immunol 137, 73-81 (2005).

Kapp, J. A., Kapp, L. M. & McKenna, K. C. Gammadelta T cells play an essential role in several forms of tolerance. Immunol Res 29, 93-102 (2004).

Karin, M., and Greten, F. R. 2005. NF-kappaB: linking inflammation and immunity to cancer development and progression. Nat Rev Immunol 5:749-759 (2005).

Ke, Y., Kapp, L. M. & Kapp, J. A. Inhibition of tumor rejection by gammadelta T cells and IL-10. Cell Immunol 221, 107-14 (2003).

Klein, L., Khazaie, K., and von Boehmer, H. 2003. In vivo dynamics of antigen-specific regulatory T cells not predicted from behavior in vitro. Proc Natl Acad Sci USA 100:8886-8891.

Kretschmer, K., Apostolou, I., Hawiger, D., Khazaie, K., Nussenzweig, M. C., and von Boehmer, H. 2005. Inducing and expanding regulatory T cell populations by foreign antigen. Nat Immunol.

Liu, H., Komai-Koma, M., Xu, D., and Liew, F. Y. 2006. Toll-like receptor 2 signaling modulates the functions of CD4⁺CD25⁺ regulatory T cells. Proc Natl Acad Sci USA 103:7048-7053 (2006).

Liyanage, U. K. et al. Prevalence of regulatory T cells is increased in peripheral blood and tumor microenvironment of patients with pancreas or breast adenocarcinoma. J Immunol 169, 2756-61 (2002).

McNeel, D. G., and Malkovsky, M. 2005. Immune-based therapies for prostate cancer. Immunol Lett 96:3-9.

Modlin, R. L. et al. Lymphocytes bearing antigen-specific gamma delta T-cell receptors accumulate in human infectious disease lesions. Nature 339, 544-8 (1989).

Morgan, M. E., van Bilsen, J. H., Bakker, A. M., Heemskerk, B., Schilham, M. W., Hartgers, F. C., Elferink, B. G., van der Zanden, L., de Vries, R. R., Huizinga, T. W., et al. 2005. Expression of FOXP3 mRNA is not confined to CD4⁺CD25⁺ T regulatory cells in humans. Hum Immunol 66:13-20.

Nishikawa, H., Kato, T., Tanida, K., Hiasa, A., Tawara, I., Ikeda, H., Ikarashi, Y., Wakasugi, H., Kronenberg, M., Nakayama, T., et al. 2003. CD4⁺ CD25⁺ T cells responding to serologically defined autoantigens suppress antitumor immune responses. Proc Natl Acad Sci USA 100:10902-10906.

Okita, R., Saeki, T., Takashima, S., Yamaguchi, Y. & Toge, T. CD4⁺CD25⁺ regulatory T cells in the peripheral blood of patients with breast cancer and non-small cell lung cancer. Oncol Rep 14, 1269-73 (2005).

Old, L. J. 1996. Immunotherapy for cancer. Sci Am 275:136-143.

Onizuka, S., Tawara, I., Shimizu, J., Sakaguchi, S., Fujita, T., and Nakayama, E. 1999. Tumor rejection by in vivo administration of anti-CD25 (interleukin-2 receptor alpha) monoclonal antibody. Cancer Res 59:3128-3133.

Peng, G., Guo, Z., Kiniwa, Y., Voo, K. S., Peng, W., Fu, T., Wang, D. Y., Li, Y., Wang, H. Y., and Wang, R.-F. 2005. Toll-like receptor 8 mediated-reversal of CD4⁺ regulatory T cell function. Science 309:1380-1384 (2005).

Pennington, D. J. et al. The integration of conventional and unconventional T cells that characterizes cell-mediated responses. Adv Immunol 87, 27-59 (2005).

Remington's Pharmaceutical Sciences, 18th Ed. Mack Printing Company, 1990.

Rifa'i, M., Kawamoto, Y., Nakashima, I., and Suzuki, H. 2004. Essential roles of CD8⁺CD122⁺ regulatory T cells in the maintenance of T cell homeostasis. J Exp Med 200:1123-1134.

Roncador, G., Brown, P. J., Maestre, L., Hue, S., Martinez-Torrecuadrada, J. L., Ling, K. L., Pratap, S., Toms, C., Fox, B. C., Cerundolo, V., et al. 2005. Analysis of FOXP3 protein expression in human CD4⁺CD25⁺ regulatory T cells at the single-cell level. Eur J Immunol 35:1681-1691.

Rosenberg, S. A. 2001. Progress in human tumour immunology and immunotherapy. Nature 411:380-384.

Rosenberg, S. A., Yang, J. C. & Restifo, N. P. Cancer immunotherapy: moving beyond current vaccines. Nat Med 10, 909-15 (2004).

Sakaguchi, S. Naturally Arising CD4⁺ Regulatory T Cells for Immunologic Self-Tolerance and Negative Control of Immune Responses. Annu Rev Immunol 22, 531-562 (2004).

Sarantopoulos, S., Lu, L., and Cantor, H. 2004. Qa-1 restriction of CD8⁺ suppressor T cells. J Clin Invest 114:1218-1221.

Sato, E., Olson, S. H., Ahn, J., Bundy, B., Nishikawa, H., Qian, F., Jungbluth, A. A., Frosina, D., Gnjatic, S., Ambrosone, C., et al. 2005. Intraepithelial CD8⁺ tumor-infiltrating lymphocytes and a high CD8⁺/regulatory T cell ratio are associated with favorable prognosis in ovarian cancer. Proc Natl Acad Sci USA 102:18538-18543.

Scotet, E. et al. Tumor recognition following Vgamma9Vdelta2 T cell receptor interactions with a surface F1-ATPase-related structure and apolipoprotein A-I. Immunity 22, 71-80 (2005).

Seo, N., Tokura, Y., Takigawa, M. & Egawa, K. Depletion of IL-10- and TGF-beta-producing regulatory gamma delta T cells by administering a daunomycin-conjugated specific monoclonal antibody in early tumor lesions augments the activity of CTLs and NK cells. J Immunol 163, 242-9 (1999).

Shevach, E. M. CD4⁺ CD25⁺ suppressor T cells: more questions than answers. Nat Rev Immunol 2, 389-400 (2002).

Shin, S. et al. Antigen recognition determinants of gammadelta T cell receptors. Science 308, 252-5 (2005).

Shiohara, T. et al. Loss of epidermal integrity by T cell-mediated attack induces long-term local resistance to subsequent attack. I. Induction of resistance correlates with increases in Thy-1⁺ epidermal cell numbers. J Exp Med 171, 1027-41 (1990).

Shiohara, T., Moriya, N., Hayakawa, J., Itohara, S. & Ishikawa, H. Resistance to cutaneous graft-vs.-host disease is not induced in T cell receptor delta gene-mutant mice. J Exp Med 183, 1483-9 (1996).

Sutmuller, R. P., den Brok, M. H., Kramer, M., Bennink, E. J., Toonen, L. W., Kullberg, B. J., Joosten, L. A., Akira, S., Netea, M. G., and Adema, G. J. 2006. Toll-like receptor 2 controls expansion and function of regulatory T cells. J Clin Invest 116:485-494 (2006).

Tien, A. H., Xu, L., and Helgason, C. D. 2005. Altered immunity accompanies disease progression in a mouse model of prostate dysplasia. Cancer Res 65:2947-2955.

Viey, E. et al. Phosphostim-activated gamma delta T cells kill autologous metastatic renal cell carcinoma. J Immunol 174, 1338-47 (2005).

Vlad, G., Cortesini, R., and Suciu-Foca, N. 2005. License to heal: bidirectional interaction of antigen-specific regulatory T cells and tolerogenic APC. J Immunol 174:5907-5914.

Wang, H. Y. & Wang, R. F. Antigen-specific CD4⁺ regulatory T cells in cancer: implications for immunotherapy. Microbes Infect 7, 1056-62 (2005).

Wang, H. Y., Deen A. Lee, Guangyong Peng, Zhong Guo, Yanchun Li, Yukiko Kiniwa, Shevach, E. M., and Wang, R.-F. 2004. Tumor-specific human CD4⁺ regulatory T cells and their ligands: implication for immunotherapy. Immunity 20:107-118 (2004).

Wang, H. Y., Peng, G., Guo, Z., Shevach, E. M., and Wang, R.-F. 2005. Recognition of a new ARTC1 peptide ligand uniquely expressed in tumor cells by antigen-specific CD4⁺ gegulatory T cells. J. Immunol. 174:2661-2670.

Wang, R. F. 2002. Enhancing antitumor immune responses: intracellular peptide delivery and identification of MHC class II-restricted tumor antigens. Immunol Rev 188:65-80.

Wang, R. F. 2006. Functional control of regulatory T cells and cancer immunotherapy. Semin Cancer Biol 16:106-114.

Wang, R. F., Peng, G., and Wang, H. Y. 2006. Regulatory T cells and Toll-like receptors in tumor immunity. Semin Immunol 18:136-142.

Woo, E. Y., Chu, C. S., Goletz, T. J., Schlienger, K., Yeh, H., Coukos, G., Rubin, S. C., Kaiser, L. R., and June, C. H. 2001. Regulatory CD4(⁺)CD25(⁺) T cells in tumors from patients with early-stage non-small cell lung cancer and late-stage ovarian cancer. Cancer Res 61:4766-4772.

Zhang, L., Conejo-Garcia, J. R., Katsaros, D., Gimotty, P. A., Massobrio, M., Regnani, G., Makrigiannakis, A., Gray, H., Schlienger, K., Liebman, M. N., et al. 2003. Intratumoral T cells, recurrence, and survival in epithelial ovarian cancer. N Engl J Med 348:203-213.

Ziegler, S. F. 2006. FOXP3: Of Mice and Men. Annu Rev Immunol 24:209-226.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method for suppressing the activity of a CD8⁺ or γδ T-regulatory cell comprising providing to the cell an effective amount of a composition capable of suppressing the activity of the T-regulatory cell, wherein the composition is not a Type D CpG oligonucleotide.

2. The method of claim 1, wherein said composition is further defined as a toll-like receptor 8 (TLR8) ligand.

3. The method of claim 1, wherein said composition is further defined as an oligonucleotide.

4. The method of claim 3, wherein the oligonucleotide is further defined as a non CpG containing oligonucleotide.

5. The method of claim 3, wherein the oligonucleotide comprises between about 4 and about 15 nucleotide residues.

6. The method of claim 3, wherein the oligonucleotide comprises between about 5 and about 10 nucleotide residues.

7. The method of claim 3, wherein the oligonucleotide comprises at least one guanine and at least one nuclease-resistant inter-residue backbone linkage.

8. The method of claims 7, wherein the oligonucleotide further comprises a nuclease-sensitive inter-residue backbone linkage.

9. The method of claim 7, wherein the oligonucleotide comprises a nuclease resistant inter-residue backbone linkage connecting the guanine to an adjacent nucleobase.

10. The method of claim 1, wherein the cell is within a subject.

11. The method of claim 10, wherein the subject is human.

12. The method of claim 8, further comprising providing the human with a therapeutic agent.

13. The method of claim 12, wherein the therapeutic agent is an anti-cancer agent, an anti-bacterial agent, or an anti-viral agent.

14. A method for suppressing the activity of at least one CD8⁺ or γδ T-regulatory cell, comprising providing to the cell an effective amount of at least one recombinant DNA capable of activating the TLR8-MyD88-IRAK4 signal transduction pathway in the cell.

15. The method of claim 14, wherein the recombinant DNA is not a type-D CpG oligonucleotide.

16. The method of claim 14, wherein the recombinant DNA is further defined as a non CpG containing recombinant DNA.

17. The method of claim 14, wherein the recombinant DNA comprises between about 4 and about 15 nucleotide residues.

18. The method of claim 14, wherein the recombinant DNA comprises between about 5 and about 10 nucleotide residues.

19. The method of claim 14, wherein the recombinant DNA comprises at least one guanine residue and at least one nuclease-resistant inter-residue backbone linkage.

20. The method of claim 19, wherein the recombinant DNA further comprises a nuclease-sensitive inter-residue backbone linkage.

21. The method of claim 19, wherein at least one guanine residue has at least one nuclease resistant inter-residue backbone linkage connecting the guanine residue with an adjacent nucleotide.

22. The method of claim 14, wherein the cell is within an organism.

23. The method of claim 22, wherein the organism is a mammal.

24. The method of claim 22, wherein the organism is human.

25. The method of claim 23, further comprising providing the human with a therapeutic agent.

26. The method of claim 25, wherein the therapeutic agent is an anti-cancer agent, an anti-bacterial agent, or an anti-viral agent.

27. A method of treating an organism with an immune-related disease, comprising administering to said organism an effective amount of at least one recombinant DNA capable of modulating or suppressing the activity of at least one CD8⁺ or γδ T-regulatory cell, thereby increasing an immune response, wherein the recombinant DNA is not a type D CpG oligonucleotide.

28. The method of claim 27, wherein said immune-related disease comprises cancer, infectious disease, or autoimmune disease.

29. The method of claim 27, wherein the recombinant DNA is further defined as a non CpG containing recombinant DNA.

30. The method of claim 27, wherein the recombinant DNA comprises between about 4 and about 15 nucleotide residues.

31. The method of claim 27, wherein the recombinant DNA comprises between about 5 and about 10 nucleotide residues.

32. The method of claim 27, wherein the recombinant DNA comprises at least one guanine residue and at least one nuclease-resistant inter-residue backbone linkage.

33. The method of claim 32, wherein at least one guanine residue has at least one nuclease resistant inter-residue backbone linkage connecting the guanine residue with an adjacent nucleotide.

34. The method of claim 27, wherein the organism is a mammal.

35. The method of claim 27, wherein the organism is human.

36. The method of claim 35, further comprising providing the human with a therapeutic agent.

37. The method of claim 36, wherein the therapeutic agent is an anti-cancer agent, an anti-bacterial agent, or an anti-viral agent.

38. A method for screening for compounds that inhibit the suppressive function of Treg cells, comprising the steps of:
a. subjecting a Treg cell to a candidate compound;
b. stimulating the proliferation of a naïve T cell;
c. exposing the naïve T cell to the Treg cell; and
d. determining the degree of growth or proliferation of the naïve T cell.

39. The method of claim 38, wherein the candidate compound is selected from a library of candidate compounds.

40. The method of claim 38, wherein the candidate compound is an oligonucleotide, a polypeptide, a polynucleotide, a small molecule, or a mixture thereof.

41. The method of claim 38, wherein the degree of proliferation is measured relative to the degree of proliferation of a control, the control consisting essentially of a Treg cell exposed to an oligonucleotide incapable of suppressing Treg cell activity.

42. The method of claim 38, wherein said candidate compound is suspected of being a TLR8 ligand.

43. A method for screening for compounds that inhibit the suppressive function of Treg cells, comprising the steps of:
providing Treg cells in the presence of naïve T cells;
subjecting said Treg cells to a candidate compound; and
assessing proliferation of the naïve T cells, wherein when there is proliferation of the naïve T cells as compared to that in the presence of the Treg cells but absence of the candidate compound, said candidate compound is said compound that inhibits suppressive function of Treg cells.

44. The method of claim 43, wherein said candidate compound is suspected of being a TLR8 ligand.

45. The method of claim 43, wherein said candidate compound is an oligonucleotide, a polypeptide, a polynucleotide, a small molecule, or a mixture thereof.


SEQUENCE LISTING (17 sequences)

SEQ ID NO: 1 - DNA, 22 bp, Artificial Sequence
  Artificial Primer
tttcccacctaccctctggctt

SEQ ID NO: 2 - DNA, 25 bp, Artificial Sequence
  Artificial Primer
tgctctgcatgaggttgtcggatga

SEQ ID NO: 3 - DNA, 20 bp, Artificial Sequence
  Artificial Primer
cgagatccctccaaaatcaa

SEQ ID NO: 4 - DNA, 20 bp, Artificial Sequence
  Artificial Primer
tgtggtcatgagtccttcca

SEQ ID NO: 5 - DNA, 20 bp, Artificial Sequence
  Artificial Oligonucleotides
aaaagacgatcgtcaaaaaa

SEQ ID NO: 6 - DNA, 10 bp, Artificial Sequence
  Artificial Oligonucleotides
gggggggggg

SEQ ID NO: 7 - DNA, 10 bp, Artificial Sequence
  Artificial Oligonucleotides
aaaaaaaaaa

SEQ ID NO: 8 - DNA, 10 bp, Artificial Sequence
  Artificial Oligonucleotides
tttttttttt

SEQ ID NO: 9 - DNA, 10 bp, Artificial Sequence
  Artificial Oligonucleotides
cccccccccc

SEQ ID NO: 10 - DNA, 20 bp, Artificial Sequence
  Artificial Oligonucleotides
  misc_feature (1)..(2): N may be any nucleobase or no nucleobase
nntgcatcgatgcagggggg

SEQ ID NO: 11 - DNA, 20 bp, Artificial Sequence
  Artificial Oligonucleotides
  misc_feature (1)..(2): N may be any nucleobase or no nucleobase
nntgcaccggtgcagggggg

SEQ ID NO: 12 - DNA, 20 bp, Artificial Sequence
  Artificial Oligonucleotides
  misc_feature (1)..(2): N may be any nucleobase or no nucleobase
nntgcgtcgacgcagggggg

SEQ ID NO: 13 - DNA, 20 bp, Artificial Sequence
  Artificial Oligonucleotides
  misc_feature (1)..(2): N may be any nucleobase or no nucleobase
nntgcgccggcgcagggggg

SEQ ID NO: 14 - DNA, 20 bp, Artificial Sequence
  Artificial Oligonucleotides
ggtgcatcgatgcagggggg

SEQ ID NO: 15 - DNA, 20 bp, Artificial Sequence
  Artificial Oligonucleotides
ggtgcgtcgacgcagggggg

SEQ ID NO: 16 - DNA, 20 bp, Artificial Sequence
  Artificial Oligonucleotides
ggtgcaccggtgcagggggg

SEQ ID NO: 17 - DNA, 19 bp, Artificial Sequence
  Artificial Oligonucleotides
ggtgcatcgatgcaggggg